(19) United States
(12) Patent Application Publication
(10) Pub. No.: US 2024/0012004 A1
(43) Pub. Date: Jan. 11, 2024

(54) INCREASED VOLTAGE-GATED SODIUM CHANNEL ALPHA PROTEIN SUBUNIT EXPRESSION THROUGH VIRAL 2A-MEDIATED CO-EXPRESSION OF NAV BETA SUBUNITS

(71) Applicant: Merck Sharp & Dohme LLC, Rahway, NJ (US)

(72) Inventors: Richard L. Kraus, Chalfont, PA (US); John Majercak, Wayne, PA (US)

(73) Assignee: Merck Sharp & Dohme LLC, Rahway, NJ (US)

(21) Appl. No.: 18/252,267

(22) PCT Filed: Nov. 12, 2021
(86) PCT No.: PCT/US2021/059044

§ 371 Date: May 9, 2023

Related U.S. Application Data
(60) Provisional application No. 63/114,861, filed on Nov. 17, 2020.

(51) Int. Cl. G01N 33/68; G01N 33/50; C07K 14/705

(52) U.S. Cl. CPC G01N 33/6872; G01N 33/502; C07K 14/705; C07K 2319/50

(57) ABSTRACT

A voltage-gated sodium channel expression system is described. The system comprises providing a polycistronic RNA message that encodes a polyprotein comprising a voltage-gated sodium channel alpha protein (Navα) subunit and one or more voltage-gated sodium channel beta protein (Navβ) subunits, each of said subunits being separated by a 2A self-cleaving peptide. During translation, the polyprotein is cleaved into individual subunit proteins which can assemble into a voltage-gated sodium channel. Host cells and lipoparticles comprising the sodium channel expression system are also provided.

### BACKGROUND OF THE INVENTION

### Field of the Invention

The present invention relates to a voltage-gated sodium channel expression system comprising a polycistronic RNA message that encodes a polyprotein that comprises a voltage-gated sodium channel alpha protein (Navα) subunit and one or more voltage-gated sodium channel accessory protein (Navβ) subunits, wherein each subunit is separated from adjacent subunits by a 2A self-cleaving peptide. During translation of the polycistronic RNA message in a host cell, the polyprotein is cleaved into individual subunit proteins, which may then assemble into a voltage-gated sodium channel integrated into a membrane of the host cell. Host cells and lipoparticles comprising the voltage-gated sodium channel expression system are also provided.

### Description of Related Art

Sodium channels are integral membrane proteins that form ion channels, conducting sodium ions (Na+) through a cell's plasma membrane. They belong to the superfamily of cation channels and can be classified according to the trigger that opens the channel for such ions, i.e., either a voltage-change (“voltage-gated”, “voltage-sensitive”, or “voltage-dependent” sodium channel; also called “VGSCs” or “Nav channel”) or a binding of a substance (a ligand) to the channel (ligand-gated sodium channels). Sodium channels comprise a large a protein (Navα) subunit that associates with accessory proteins, such as β protein (Navβ) subunits. Navα subunits form the core of the channel which is functional on its own, thus when the Navα subunit is expressed by a cell, it is able to form channels that conduct Na⁺ in a voltage-gated manner, even when Navβ subunits or other known modulating proteins are absent. However, when accessory proteins such as Navβ subunits assemble with Navα subunits, the resulting complex can display altered voltage dependence and cellular localization.

Navα subunits have four repeat domains, DI through DIV, each containing six membrane-spanning segments, labelled S1 through S6 (See FIG. 1 and FIG. 2). The highly conserved S4 segment acts as the channel's voltage sensor. The voltage sensitivity is due to positive amino acids located at every third position. When stimulated by a change in transmembrane voltage, this segment moves toward the extracellular side of the cell membrane, thereby allowing the channel to become permeable to ions. The ions are conducted through a pore comprising a more extracellular portion of the pore that is formed by the “P-loops” (the region between S5 and S6) of the four domains. This region is the narrowest part of the pore and is responsible for its ion selectivity. The more cytoplasmic portion of the pore is formed by the combined S5 and S6 segments of the four domains. The region linking domains III and IV is also important for channel function. This region plugs the channel after prolonged activation thereby inactivating it.

There are nine known Navα subunits of the super family, which are named Nav1.1α, Nav1.2α, Nav1.3α, Nav1.4α, Nav1.5α, Nav1.6α, Nav1.7α, Nav1.8α, and Nav1.9α (genes SCN1A, SCN2A, SCN3A, SCN4A, SCN5A, SCN8A, SCN9A, SCN10A, and SCN11A, respectively) distinguished not only by differences in their amino acid sequence but also by their kinetics and expression profiles. These Navα subunits have greater than 50% amino acid sequence between them in the transmembrane portions and extracellular loop regions.

Sodium channel Navβ subunits are type 1 transmembrane glycoproteins with an extracellular N-terminus and a cytoplasmic C-terminus. As members of the Ig superfamily, Navβ subunits contain a prototypic V-set Ig loop in their extracellular domain. They are homologous to neural cell adhesion molecules (CAMs) and the large family of L1 CAMs. There are four distinct Navβ subunits named in order of discovery: gene SCN1B encoding Navβ1, gene SCN2B encoding Navβ2, gene SCN3B encoding Navβ3, and gene SCN4B encoding Navβ4. Navβ1 and Navβ3 subunits interact with Navα subunits non-covalently, whereas Navβ2 and Navβ4 subunits associate with Navα subunits via disulfide bond. Sodium channels are more likely to stay open at the subthreshold membrane potential when interacting with β toxins, which in turn induces an immediate sensation of pain.

In addition to regulating channel gating, Navβ subunits also modulate channel expression and form links to the intracellular cytoskeleton via ankyrin and spectrin.

Protein reagents and cell lines that express Navα subunits for screening drug-like molecules exist; however, in general these reagents are limited in total and/or cell surface and/or functional expression. Currently, expression of Navα subunits have proven to be limiting due to several likely factors such as large size (−225 kDa), interaction with known auxiliary proteins such as Navβ subunits, extensive post-translational modification, and regulated cell surface expression. It is desirable if reagents could be engineered to improve expression of the Navα and Navβ subunits simultaneously in a host cell, which can then form a functional voltage-gated sodium channels in the host cells or in lipoparticles, such host cells may serve as an advantageous tool for discovery research.

### BRIEF SUMMARY OF THE INVENTION

The present invention provides a voltage-gated sodium channel expression system that comprises a polynucleotide encoding a polycistronic RNA message comprising an open reading frame (ORF) encoding a polyprotein comprising a voltage-gated sodium channel a protein (Navα) subunit and one or more voltage-gated sodium channel β protein (Navβ) subunits wherein the polycistronic RNA message further encodes a cleavage peptide located between adjacent subunits, which upon cleavage, produces a Navα subunit and one or more of the Navβ subunits, which are capable of assembling into a voltage-gated sodium channel. In specific embodiment, the subunits are in tandem. As used herein the Navα subunit may be selected from the group consisting of Nav1.1α, Nav1.2α, Nav1.3α, Nav1.4α, Nav1.5α, Nav1.6α, Nav1.7α, Nav1.8α, and Nav1.9α subunits and the Navβ subunits may be selected from the group consisting of Navβ1, Navβ2, Navβ3, and Navβ4 subunits with the proviso that the polyprotein does not encode more than one copy of any particular Navβ subunit. Upon translation of the polycistronic RNA message, a polyprotein is produced, which upon cleavage of the cleavage peptide, produces individual Navα and Navβ subunits, which can assemble into a voltage-gated sodium channel. In particular embodiments, the cleavage peptide is a viral 2A self-cleaving peptide including, but not limited to, the viral P2A peptide from porcine teschovirus-1 2A, the viral T2A peptide from thosea asigna virus 2A, the viral E2A peptide from equine rhinitis A virus, or the viral F2A peptide from foot-and-mouth disease virus 18.

In a particular embodiment of the present invention, the polycistronic message may encode a polyprotein comprising a Navα subunit and one or more Navβ subunits having a structure according to:
(i) Nav1.1α-2A-Navβ1; Nav1.1α-2A-Navβ2; Nav1.1α-2A-Navβ3; Nav1.1α-2A-Navβ4; Nav1.1α-2A-Navβ1-2A-Navβ2; Nav1.1α-2A-Navβ1-2A-Navβ3; Nav1.1α-2A-Navβ1-2A-Navβ4; Nav1.1α-2A-Navβ2-2A-Navβ3; Nav1.1α-2A-Navβ1-2A-Navβ4; Nav1.1α-2A-Navβ3-2A-Navβ4; or, Nav1.1α-2A-Navβ1-2A-Navβ2-2A-β3; Nav1.1α-2A-Navβ1-2A-Navβ2-2A-β4; or, Nav1.1α-2A-Navβ1-2A-Navβ2-2A-β3-2A-Navβ2-2A-β4;
(ii) Nav1.2α-2A-Navβ1; Nav1.2α-2A-Navβ2; Nav1.2α-2A-Navβ3; Nav1.2α-2A-Navβ4; Nav1.2α-2A-Navβ1-2A-Navβ2; Nav1.2α-2A-Navβ1-2A-Navβ3; Nav1.2α-2A-Navβ1-2A-Navβ4; Nav1.2α-2A-Navβ2-2A-Navβ3; Nav1.2α-2A-Navβ1-2A-Navβ4; Nav1.2α-2A-Navβ3-2A-Navβ4; or, Nav1.2α-2A-Navβ1-2A-Navβ2-2A-β3; Nav1.2α-2A-Navβ1-2A-Navβ2-2A-β4; or, Nav1.2α-2A-Navβ1-2A-Navβ2-2A-β3-2A-Navβ2-2A-β4;
(iii) Nav1.3α-2A-Navβ1; Nav1.3α-2A-Navβ2; Nav1.3α-2A-Navβ3; Nav1.3α-2A-Navβ4; Nav1.3α-2A-Navβ1-2A-Navβ2; Nav1.3α-2A-Navβ1-2A-Navβ3; Nav1.3α-2A-Navβ1-2A-Navβ4; Nav1.3α-2A-Navβ2-2A-Navβ3; Nav1.3α-2A-Navβ1-2A-Navβ4; Nav1.3α-2A-Navβ3-2A-Navβ4; or, Nav1.3α-2A-Navβ1-2A-Navβ2-2A-β3; Nav1.3α-2A-Navβ1-2A-Navβ2-2A-β4; or, Nav1.3α-2A-Navβ1-2A-Navβ2-2A-β3-2A-Navβ2-2A-β4;
(iv) Nav1.4α-2A-Navβ1; Nav1.4α-2A-Navβ2; Nav1.4α-2A-Navβ3; Nav1.4α-2A-Navβ4; Nav1.4α-2A-Navβ1-2A-Navβ2; Nav1.4α-2A-Navβ1-2A-Navβ3; Nav1.4α-2A-Navβ1-2A-Navβ4; Nav1.4α-2A-Navβ2-2A-Navβ3; Nav1.4α-2A-Navβ1-2A-Navβ4; Nav1.4α-2A-Navβ3-2A-Navβ4; or, Nav1.4α-2A-Navβ1-2A-Navβ2-2A-β3; Nav1.4α-2A-Navβ1-2A-Navβ2-2A-β4; or, Nav1.4α-2A-Navβ1-2A-Navβ2-2A-β3-2A-Navβ2-2A-β4;
(v) Nav1.5α-2A-Navβ1; Nav1.5α-2A-Navβ2; Nav1.5α-2A-Navβ3; Nav1.5α-2A-Navβ4; Nav1.5α-2A-Navβ1-2A-Navβ2; Nav1.5α-2A-Navβ1-2A-Navβ3; Nav1.5α-2A-Navβ1-2A-Navβ4; Nav1.5α-2A-Navβ2-2A-Navβ3; Nav1.5α-2A-Navβ1-2A-Navβ4; Nav1.5α-2A-Navβ3-2A-Navβ4; or, Nav1.5α-2A-Navβ1-2A-Navβ2-2A-β3; Nav1.5α-2A-Navβ1-2A-Navβ2-2A-β4; or, Nav1.5α-2A-Navβ1-2A-Navβ2-2A-β3-2A-Navβ2-2A-β4;
(vi) Nav1.6α-2A-Navβ1, Nav1.6α-2A-Navβ2; Nav1.6α-2A-Navβ3; Nav1.6α-2A-Navβ4; Nav1.6α-2A-Navβ1-2A-Navβ2; Nav1.6α-2A-Navβ1-2A-Navβ3; Nav1.6α-2A-Navβ1-2A-Navβ4; Nav1.6α-2A-Navβ2-2A-Navβ3; Nav1.6α-2A-Navβ1-2A-Navβ4; Nav1.6α-2A-Navβ3-2A-Navβ4; or, Nav1.6α-2A-Navβ1-2A-Navβ2-2A-β3; Nav1.6α-2A-Navβ1-2A-Navβ2-2A-β4; or, Nav1.6α-2A-Navβ1-2A-Navβ2-2A-β3-2A-Navβ2-2A-β4;
(vii) Nav1.7α-2A-Navβ1; Nav1.7α-2A-Navβ2; Nav1.7α-2A-Navβ3; Nav1.7α-2A-Navβ4; Nav1.7α-2A-Navβ1-2A-Navβ2; Nav1.7α-2A-Navβ1-2A-Navβ3; Nav1.7α-2A-Navβ1-2A-Navβ4; Nav1.7α-2A-Navβ2-2A-Navβ3; Nav1.7α-2A-Navβ1-2A-Navβ4; Nav1.7α-2A-Navβ3-2A-Navβ4; or, Nav1.7α-2A-Navβ1-2A-Navβ2-2A-β3; Nav1.7α-2A-Navβ1-2A-Navβ2-2A-β4; or, Nav1.7α-2A-Navβ1-2A-Navβ2-2A-β3-2A-Navβ2-2A-β4;
(viii) Nav1.8α-2A-Navβ1; Nav1.8α-2A-Navβ2; Nav1.8α-2A-Navβ3; Nav1.8α-2A-Navβ4; Nav1.8α-2A-Navβ1-2A-Navβ2; Nav1.8α-2A-Navβ1-2A-Navβ3; Nav1.8α-2A-Navβ1-2A-Navβ4; Nav1.8α-2A-Navβ2-2A-Navβ3; Nav1.8α-2A-Navβ1-2A-Navβ4; Nav1.8α-2A-Navβ3-2A-Navβ4; or, Nav1.8α-2A-Navβ1-2A-Navβ2-2A-β3; Nav1.8α-2A-Navβ1-2A-Navβ2-2A-β4; or, Nav1.8α-2A-Navβ1-2A-Navβ2-2A-β3-2A-Navβ2-2A-β4; or
(ix) Nav1.9α-2A-Navβ1; Nav1.9α-2A-Navβ2; Nav1.9α-2A-Navβ3; Nav1.9α-2A-Navβ4; Nav1.9α-2A-Navβ1-2A-Navβ2; Nav1.9α-2A-Navβ1-2A-Navβ3; Nav1.9α-2A-Navβ1-2A-Navβ4; Nav1.9α-2A-Navβ2-2A-Navβ3; Nav1.9α-2A-Navβ1-2A-Navβ4; Nav1.9α-2A-Navβ3-2A-Navβ4; or, Nav1.9α-2A-Navβ1-2A-Navβ2-2A-β3; Nav1.9α-2A-Navβ1-2A-Navβ2-2A-β4; or, Nav1.9α-2A-Navβ1-2A-Navβ2-2A-β3-2A-Navβ2-2A-β4;
   wherein 2A is a viral peptide selected from the group consisting of P2A, T2A, E2A, F2A.

In a particular embodiments of the present invention, the polycistronic message may encode a polyprotein comprising a Navα subunit and one or more Navβ subunits having a structure according to:
(i) Nav1.1α-PP2A-Navβ1; Nav1.1α-P2A-Navβ2; Nav1.1α-P2A-Navβ3; Nav1.1α-P2A-Navβ4; Nav1.1α-P2A-Navβ1-P2A-Navβ2; Nav1.1α-P2A-Navβ1-P2A-Navβ3; Nav1.1α-P2A-Navβ1-P2A-Navβ4; Nav1.1α-P2A-Navβ2-P2A-Navβ3; Nav1.1α-P2A-Navβ1-P2A-Navβ4; Nav1.1α-P2A-Navβ3-P2A-Navβ4; or, Nav1.1α-P2A-Navβ1-P2A-Navβ2-P2A-β3; Nav1.1α-P2A-Navβ1-P2A-Navβ2-P2A-β4; or, Nav1.1α-P2A-Navβ1-P2A-Navβ2-P2A-β3-P2A-Navβ2-P2A-β4;
(ii) Nav1.2α-P2A-Navβ1; Nav1.2α-P2A-Navβ2; Nav1.2α-P2A-Navβ3; Nav1.2α-P2A-Navβ4; Nav1.2α-P2A-Navβ1-P2A-Navβ2; Nav1.2α-P2A-Navβ1-P2A-Navβ3; Nav1.2α-P2A-Navβ1-P2A-Navβ4; Nav1.2α-P2A-Navβ2-P2A-Navβ3; Nav1.2α-P2A-Navβ1-P2A-Navβ4; Nav1.2α-P2A-Navβ3-P2A-Navβ4; or, Nav1.2α-P2A-Navβ1-P2A-Navβ2-P2A-β3; Nav1.2α-P2A-Navβ1-P2A-Navβ2-P2A-β4; or, Nav1.2α-P2A-Navβ1-P2A-Navβ2-P2A-β3-P2A-Navβ2-P2A-β4;
(iii) Nav1.3α-P2A-Navβ1; Nav1.3α-P2A-Navβ2; Nav1.3α-P2A-Navβ3; Nav1.3α-P2A-Navβ4; Nav1.3α-P2A-Navβ1-P2A-Navβ2; Nav1.3α-P2A-Navβ1-P2A-Navβ3; Nav1.3α-P2A-Navβ1-P2A-Navβ4; Nav1.3α-P2A-Navβ2-P2A-Navβ3; Nav1.3α-P2A-Navβ1-P2A-Navβ4; Nav1.3α-P2A-Navβ3-P2A-Navβ4; or, Nav1.3α-P2A-Navβ1-P2A-Navβ2-P2A-β3; Nav1.3α-P2A-Navβ1-P2A-Navβ2-P2A-β4; or, Nav1.3α-P2A-Navβ1-P2A-Navβ2-P2A-β3-P2A-Navβ2-P2A-β4;
(iv) Nav1.4α-P2A-Navβ1; Nav1.4α-P2A-Navβ2; Nav1.4α-P2A-Navβ3; Nav1.4α-P2A-Navβ4; Nav1.4α-P2A-Navβ1-P2A-Navβ2; Nav1.4α-P2A-Navβ1-P2A-Navβ3; Nav1.4α-P2A-Navβ1-P2A-Navβ4; Nav1.4α-P2A-Navβ2-P2A-Navβ3; Nav1.4α-P2A-Navβ1-P2A-Navβ4; Nav1.4α-P2A-Navβ3-P2A-Navβ4; or, Nav1.4α-P2A-Navβ1-P2A-Navβ2-P2A-β3; Nav1.4α-P2A-Navβ1-P2A-Navβ2-P2A-β4; or, Nav1.4α-P2A-Navβ1-P2A-Navβ2-P2A-β3-P2A-Navβ2-P2A-β4;
(v) Nav1.5α-P2A-Navβ1; Nav1.5α-P2A-Navβ2; Nav1.5α-P2A-Navβ3; Nav1.5α-P2A-Navβ4; Nav1.5α-P2A-Navβ1-P2A-Navβ2; Nav1.5α-P2A-Navβ1-P2A-Navβ3; Nav1.5α-P2A-Navβ1-P2A-Navβ4; Nav1.5α-P2A-Navβ2-P2A-Navβ3; Nav1.5α-P2A-Navβ1-P2A-Navβ4; Nav1.5α-P2A-Navβ3-P2A-Navβ4; or, Nav1.5α-P2A-Navβ1-P2A-Navβ2-P2A-β3; Nav1.5α-P2A-Navβ1-P2A-Navβ2-P2A-β4; or, Nav1.5α-P2A-Navβ1-P2A-Navβ2-P2A-β3-P2A-Navβ2-P2A-β4;
(vi) Nav1.6α-P2A-Navβ1; Nav1.6α-P2A-Navβ2; Nav1.6α-P2A-Navβ3; Nav1.6α-P2A-Navβ4; Nav1.6α-P2A-Navβ1-P2A-Navβ2; Nav1.6α-P2A-Navβ1-P2A-Navβ3; Nav1.6α-P2A-Navβ1-P2A-Navβ4; Nav1.6α-P2A-Navβ2-P2A-Navβ3; Nav1.6α-P2A-Navβ1-P2A-Navβ4; Nav1.6α-P2A-Navβ3-P2A-Navβ4; or, Nav1.6α-P2A-Navβ1-P2A-Navβ2-P2A-β3; Nav1.6α-P2A-Navβ1-P2A-Navβ2-P2A-β4; or, Nav1.6α-P2A-Navβ1-P2A-Navβ2-P2A-β3-P2A-Navβ2-P2A-β4;
(vii) Nav1.7α-P2A-Navβ1; Nav1.7α-P2A-Navβ2; Nav1.7α-P2A-Navβ3; Nav1.7α-P2A-Navβ4; Nav1.7α-P2A-Navβ1-P2A-Navβ2; Nav1.7α-P2A-Navβ1-P2A-Navβ3; Nav1.7α-P2A-Navβ1-P2A-Navβ4; Nav1.7α-P2A-Navβ2-P2A-Navβ3; Nav1.7α-P2A-Navβ1-P2A-Navβ4; Nav1.7α-P2A-Navβ3-P2A-Navβ4; or, Nav1.7α-P2A-Navβ1-P2A-Navβ2-P2A-β3; Nav1.7α-P2A-Navβ1-P2A-Navβ2-P2A-β4; or, Nav1.7α-P2A-Navβ1-P2A-Navβ2-P2A-β3-P2A-Navβ2-P2A-β4;
(viii) Nav1.8α-P2A-Navβ1; Nav1.8α-P2A-Navβ2; Nav1.8α-P2A-Navβ3; Nav1.8α-P2A-Navβ4; Nav1.8α-P2A-Navβ1-P2A-Navβ2; Nav1.8α-P2A-Navβ1-P2A-Navβ3; Nav1.8α-P2A-Navβ1-P2A-Navβ4; Nav1.8α-P2A-Navβ2-P2A-Navβ3; Nav1.8α-P2A-Navβ1-P2A-Navβ4; Nav1.8α-P2A-Navβ3-P2A-Navβ4; or, Nav1.8α-P2A-Navβ1-P2A-Navβ2-P2A-β3; Nav1.8α-P2A-Navβ1-P2A-Navβ2-P2A-β4; or, Nav1.8α-P2A-Navβ1-P2A-Navβ2-P2A-β3-P2A-Navβ2-P2A-β4; or
(ix) Nav1.9α-P2A-Navβ1; Nav1.9α-P2A-Navβ2; Nav1.9α-P2A-Navβ3; Nav1.9α-P2A-Navβ4; Nav1.9α-P2A-Navβ1-P2A-Navβ2; Nav1.9α-P2A-Navβ1-P2A-Navβ3; Nav1.9α-P2A-Navβ1-P2A-Navβ4; Nav1.9α-P2A-Navβ2-P2A-Navβ3; Nav1.9α-P2A-Navβ1-P2A-Navβ4; Nav1.9α-P2A-Navβ3-P2A-Navβ4; or, Nav1.9α-P2A-Navβ1-P2A-Navβ2-P2A-β3; Nav1.9α-P2A-Navβ1-P2A-Navβ2-P2A-β4; or, Nav1.9α-P2A-Navβ1-P2A-Navβ2-P2A-β3-P2A-Navβ2-P2A-β4.

The Navα subunit comprising the present invention may be encoded by a nucleic acid molecule comprising a nucleotide sequence selected from the group of nucleotide sequences consisting of SEQ ID NOs: 7, 9, 11, 13, 15, 17, 19, 21, and 87 and or a nucleic acid molecule sequence has at least 80%, or in specific embodiments 90%, identity to a nucleotide sequence selected from the group of nucleotide sequences consisting of SEQ ID NOs: 7, 9, 11, 13, 15, 17, 19, 21, and 87.

The Navβ subunit comprising the present invention may be encoded by a nucleic acid molecule comprising a nucleotide sequence selected from the group of nucleotide sequences consisting of SEQ ID NOs: 23, 25, 27, and 89 or a nucleic acid molecule sequence has at least 80%, or in specific embodiments 90%, identity to a nucleotide sequence selected from the group of nucleotide sequences consisting of SEQ ID NOs: 23, 25, 27, and 89.

In particular embodiments, the cleavage peptide is the viral P2A peptide from porcine teschovirus-1 2A.

In an embodiment of the present invention, a polynucleotide is provided that encodes a polyprotein comprising an amino acid sequence selected from the group consisting of SEQ ID Nos: 34, 35, 36, 37, 38, 39, 40, 41, 42, 43, 44, 45, 46, 47, 48, 49, 50, 51, 52, 53, 54, 55, 56, and 57. In particular embodiments, the polyprotein has 80%, or in specific embodiments 90%, identity to a polyprotein comprising an amino acid sequence selected from the group of amino acid sequences consisting of SEQ ID Nos: 34, 35, 36, 37, 38, 39, 40, 41, 42, 43, 44, 45, 46, 47, 48, 49, 50, 51, 52, 53, 54, 55, 56, and 57. In a further embodiment of the present invention, the polyprotein is encoded by a polynucleotide in which the ORF encoding the polyprotein comprises a nucleotide sequence selected from the group consisting of SEQ ID Nos: 58, 59, 60, 61, 62, 63, 64, 65, 66, 67, 68, 69, 70, 71, 72, 73, 74, 75, 76, 77, 78, 79, 80, and 81. In a further embodiment, the polynucleotide sequence comprises an ORF that has at least 80%, or in specific embodiments 90%, identity to a nucleotide sequence selected from the group of nucleotide sequences consisting of SEQ ID NOs: 58, 59, 60, 61, 62, 63, 64, 65, 66, 67, 68, 69, 70, 71, 72, 73, 74, 75, 76, 77, 78, 79, 80, and 81. In a further embodiment of any one of the above nucleic acid molecules or polynucleotide sequences, the nucleotide sequence is codon-optimized for expression nucleic acid molecule in mammalian or human cells. In particular embodiments, the polynucleotide encoding the expression system is operably linked to a transcriptionally active promoter, which may include one or more enhancer elements, at the 5′ end and transcription termination sequences at the 3′ end. The ORF encoding the polyprotein located within the polynucleotide is operably linked at its 5′ end to RNA translation regulatory elements and to one or more stop codons at its 3′ end.

During translation of the polycistronic RNA message, the polyprotein as it is being produced is cleaved at a cleavage site within the cleavage peptide to produce a first polypeptide comprising the portion of the cleavage peptide upstream of the cleavage site within the cleavage peptide (upstream portion) at the C-terminus of the first polypeptide and a second polypeptide comprising the portion of the cleavage peptide downstream of the cleavage site (downstream portion) at the N-terminus of the second polypeptide.

Thus, the present invention provides (a) a human Nav1.1α, Nav1.2α, Nav1.3α, Nav1.4α, Nav1.5α, Nav1.6α, Nav1.7α, Nav1.8α, or Nav1.9α subunit comprising the upstream portion of the cleavage peptide at the C-terminus; a human Nav1.1α, Nav1.2α, Nav1.3α, Nav1.4α, Nav1.5α, Nav1.6α, Nav1.7α, Nav1.8α, or Nav1.9α subunit comprising the downstream portion of the cleavage peptide at the N-terminus; or a human Nav1.1α, Nav1.2α, Nav1.3α, Nav1.4α, Nav1.5α, Nav1.6α, Nav1.7α, Nav1.8α, or Nav1.9α subunit comprising the downstream portion of the cleavage peptide at its N-terminus and comprising the upstream portion of the cleavage peptide at the C-terminus; and (b) at least one human Navβ1, Navβ2, Navβ3, or Navβ4 subunit comprising the upstream portion of the cleavage peptide at the C-terminus; a human Navβ1, Navβ2, Navβ3, or Navβ4 subunit comprising the downstream portion of the cleavage peptide at the N-terminus; or a human Navβ1, Navβ2, Navβ3, or Navβ4 subunit comprising the downstream portion of the cleavage peptide at its N-terminus and comprising the upstream portion of the cleavage peptide at the C-terminus, with the proviso that with the proviso that only one Navα or Navβ subunit comprises solely the downstream portion of the cleavage site at the N-terminus and only one Navα or Navβ subunit comprises solely the upstream portion of the peptide at the C-terminus.

In embodiments in which the cleavage peptide is a viral 2A peptide comprising a glycine-proline (GP) cleavage site wherein the GP is cleaved to provide an upstream peptide ending with a C-terminal G attached to the C-terminus of the Navα or Navβ subunit and a P and downstream portion of the viral 2A peptide having zero to 40 amino acids is at the N-terminus of the Navα or Navβ subunit, thus the present invention further provides (a) a human Nav1.1α, Nav1.2α, Nav1.3α, Nav1.4α, Nav1.5α, Nav1.6α, Nav1.7α, Nav1.8α, or Nav1.9α subunit comprising the peptide amino acid sequence upstream of the GP cleavage site at the C-terminus; a human Nav1.1α, Nav1.2α, Nav1.3α, Nav1.4α, Nav1.5α, Nav1.6α, Nav1.7α, Nav1.8α, or Nav1.9α subunit comprising a P and downstream amino acid sequence of zero to 40 amino acids at the N-terminus; or a human Nav1.1α, Nav1.2α, Nav1.3α, Nav1.4α, Nav1.5α, Nav1.6α, Nav1.7α, Nav1.8α, or Nav1.9α subunit comprising a P and downstream amino acid sequence of zero to 40 amino acids at the N-terminus and comprising the amino acid sequence upstream of the GP cleavage site at the C-terminus; and, (b) at least one human Navβ1, Navβ2, Navβ3, or Navβ4 subunit comprising the amino acid sequence upstream of the GP cleavage site at the C-terminus; a human Navβ1, Navβ2, Navβ3, or Navβ4 subunit comprising a P and downstream amino acid sequence of zero to 40 amino acids at the N-terminus; or a human Navβ1, Navβ2, Navβ3, or Navβ4 subunit comprising a P and downstream sequence of zero to 40 amino acids at the N-terminus and comprising amino acid sequence upstream of the GP cleavage site at the C-terminus; wherein only one Navα or Navβ subunit comprises solely a P and a downstream peptide of zero to 40 amino acids at the N-terminus and only one Navα or Navβ subunit comprises solely an upstream peptide at its C-terminus.

In embodiments in which the cleavage peptide is a viral P2A peptide comprising a GP cleavage site, the present invention further provides (a) a human Nav1.1α, Nav1.2α, Nav1.3α, Nav1.4α, Nav1.5α, Nav1.6α, Nav1.7α, Nav1.8α, or Nav1.9α subunit comprising the amino acid sequence GSGATNFSLLKQAGDVEENPG (SEQ ID NO: 85) at the C-terminus; a human Nav1.1α, Nav1.2α, Nav1.3α, Nav1.4α, Nav1.5α, Nav1.6α, Nav1.7α, Nav1.8α, or Nav1.9α subunit comprising a P at the N-terminus; or a human Nav1.1α, Nav1.2α, Nav1.3α, Nav1.4α, Nav1.5α, Nav1.6α, Nav1.7α, Nav1.8α, or Nav1.9α subunit comprising a P at the N-terminus and comprising the amino acid sequence GSGATNFSLLKQAGDVEENPG (SEQ ID NO: 85) at the C-terminus and (b) at least one human Navβ1, Navβ2, Navβ3, or Navβ4 subunit comprising the amino acid sequence GSGATNFSLLKQAGDVEENPG (SEQ ID NO: 85) at the C-terminus; a human Navβ1, Navβ2, Navβ3, or Navβ4 subunit comprising a P at the N-terminus; or a human Navβ1, Navβ2, Navβ3, or Navβ4 subunit comprising a P at the N-terminus and comprising the amino acid sequence GSGATNFSLLKQAGDVEENPG (SEQ ID NO: 85) at the C-terminus; wherein only one Navα or Navβ subunit comprises solely a P at the N-terminus and at only one Navα or Navβ subunit comprises solely an upstream peptide at its C-terminus.

In a further embodiment, the present invention provides a voltage-gated sodium channel comprising a human Nav1.1α, Nav1.2α, Nav1.3α, Nav1.4α, Nav1.5α, Nav1.6α, Nav1.7α, Nav1.8α, or Nav1.9α subunit comprising the upstream portion of a cleavage peptide at the C-terminus and/or a downstream portion of a cleavage peptide at the N-terminus; and, one or more of a human Navβ1 subunit comprising a downstream portion of a cleavage peptide at the N-terminus and/or the upstream portion of a cleavage peptide at the C-terminus, a human Navβ2 subunit comprising a downstream portion of a cleavage peptide at the N-terminus and/or the upstream portion of a cleavage peptide at the C-terminus, and a human Navβ3 subunit comprising a downstream portion of a cleavage peptide at the N-terminus and/or the upstream portion of a cleavage peptide at the C-terminus, with the proviso that a first subunit comprises only a downstream portion of the cleavage peptide at the N-terminus and a second subunit comprises only the upstream portion of the cleavage peptide at the C-terminus, and third and/or fourth subunits, if present, comprise a downstream portion of the cleavage peptide at the N-terminus and/or the upstream portion of the cleavage peptide at the C-terminus.

In a further embodiment, the present invention provides a voltage-gated sodium channel comprising a human Nav1.1α, Nav1.2α, Nav1.3α, Nav1.4α, Nav1.5α, Nav1.6α, Nav1.7α, Nav1.8α, or Nav1.9α subunit comprising the amino acid sequence upstream of a GP cleavage site in a 2A cleavage peptide at the C-terminus and/or comprising a P at the N-terminus; and, one or more of a human Navβ1 subunit comprising a P at the N-terminus and/or the amino acid sequence upstream of the GP cleavage site at the C-terminus, a human Navβ2 subunit comprising a P at the N-terminus and/or the amino acid sequence upstream of the GP cleavage site at the C-terminus, and a human Navβ3 subunit comprising P at the N-terminus and/or the amino acid sequence upstream of the GP cleavage site at the C-terminus, with the proviso that a first subunit comprises only a P at the N-terminus and a second subunit comprises only the amino acid sequence upstream of the GP cleavage site at the C-terminus, and third and/or fourth subunits, if present, comprise a P at the N-terminus and/or the amino acid sequence upstream of the GP cleavage site at the C-terminus.

In a further embodiment, the present invention provides a voltage-gated sodium channel comprising a human Nav1.1α, Nav1.2α, Nav1.3α, Nav1.4α, Nav1.5α, Nav1.6α, Nav1.7α, Nav1.8α, or Nav1.9α subunit comprising the amino acid sequence GSGATNFSLLKQAGDVEENPG (SEQ ID NO: 85) at the C-terminus and/or a P at the N-terminus; and, one or more of a human Navβ1 comprising a P at the N-terminus and/or comprising the amino acid sequence GSGATNFSLLKQAGDVEENPG (SEQ ID NO: 85) at the C-terminus, a human Navβ2 subunit comprising a P at the N-terminus and/or comprising the amino acid sequence GSGATNFSLLKQAGDVEENPG (SEQ ID NO: 85) at the C-terminus, and a human Navβ3 subunit comprising a P at its N-terminus and/or comprising the amino acid sequence GSGATNFSLLKQAGDVEENPG (SEQ ID NO: 85) at the C-terminus, with the proviso that a first subunit comprises only a P at its the N-terminus and a second subunit comprises only the amino acid sequence GSGATNFSLLKQAGDVEENPG (SEQ ID NO: 85) at the C-terminus, and third and/or fourth subunits, if present, comprise a P at the N-terminus and the amino acid sequence GSGATNFSLLKQAGDVEENPG (SEQ ID NO: 85) at the C-terminus.

In a further embodiment of the present invention, the human Nav1.1α, Nav1.2α, Nav1.3α, Nav1.4α, Nav1.5α, Nav1.6, Nav1.7α, or Nav1.8 subunit comprise the amino acid sequence set forth in SEQ ID NO: 6, 8, 10, 12, 14, 16, 18, or 20, respectively. In particular embodiments, the human Nav1.1α, Nav1.2α, Nav1.3α, Nav1.4α, Nav1.5α, Nav1.6, Nav1.7α, or Nav1.8 subunit comprise an amino acid sequence that has at least about 80-99.9% (in specific embodiments, 80, 81, 82, 83, 84, 85, 86, 87, 88, 89, 90, 91, 92, 93, 94, 95, 96, 97, 98, 99, 99.5, or 99.9%) identity to an amino acid sequence set forth in SEQ ID NO: 6, 8, 10, 12, 14, 16, 18, and 20, respectively.

In a further embodiment of the present invention, the human Navβ1, Navβ2, or Navβ3 subunit comprise the amino acid sequence set forth in SEQ ID NO: 22, 24, and 26, respectively. In particular embodiments, the the human Navβ1, Navβ2, or Navβ3 subunit comprise an amino acid sequence that has at least about 80-99.9% (in specific embodiments, 80, 81, 82, 83, 84, 85, 86, 87, 88, 89, 90, 91, 92, 93, 94, 95, 96, 97, 98, 99, 99.5, or 99.9%) identity to an amino acid sequence set forth in SEQ ID NO: 22, 24, and 26, respectively.

The present invention also provides an isolated host cell comprising the voltage-gated sodium channel expression system. In the host cell, expression of the polynucleotide produces a polycistronic RNA message, which during translation produces a polyprotein that is cleaved at the cleavage peptides to produce the encoded Navα subunit and the one or more Navβ subunits, which are capable of assembling into a voltage-gated sodium channel in the host cell membrane. The host cells may be disrupted using common techniques such as mechanical disruption or mild detergent treatment to produce a disrupted cell lysate from which a membrane fraction thereof comprising the assembled sodium channel may be recovered.

Thus, the present invention also provides a method of expressing a voltage-gated sodium channel comprising introducing a voltage-gated sodium channel expression system disclosed herein into a host cell and culturing the host cell under conditions favorable to expression of the voltage-gated sodium channel expression system to produce Navα subunit and one or more Navβ subunits assembled into a sodium channel in the host cell plasma membrane; and, optionally, isolating plasma membrane fractions comprising the voltage-gated sodium channel by disrupting the host cell to provide an extract or lysate, and isolating the plasma membrane fraction, which includes the voltage-gated sodium channel in the plasma membrane, from the lysate.

In particular embodiments, the voltage-gated sodium channel expression system may be performed in host cells under conditions that favor production of lipoparticles comprising assembled voltage-gated sodium channels. Thus, the present invention further provides a method for making a lipoparticle comprising an assembled voltage-gated sodium channel on the surface of the lipoparticle comprising introducing a viral vector comprising a voltage-gated sodium channel expression system disclosed herein into an isolated host cell, culturing the host cell under conditions favorable to generation of lipoparticles having assembled voltage-gated sodium channels on the surface thereof, and isolating the lipoparticles from the host cells and/or host cell culture medium. Accordingly, the present invention further provides a a lipoparticle comprising an external lipid bilayers an enveloped retroviral structural protein; and a voltage-gated sodium channel wherein said enveloped retroviral structural protein is an uncleaned gag protein, wherein said gag protein does not comprise a heterologous tag that binds to the voltage-gated sodium channel, provided that the only viral proteins in the lipoparticle are structural proteins.

The present invention also provides a method for increasing expression levels of a voltage-gated Navα subunit selected from Nav1.1α, Nav1.2α, Nav1.3α, Nav1.4α, Nav1.5α, Nav1.6α, Nav1.7α, Nav1.8α, or Nav1.9α subunits in a host cell comprising co-expressing with the Navα subunit one or more voltage-gated sodium channel Navβ subunits selected from the group consisting Navβ1, Navβ2, or Navβ3 in a host cell.

The present invention also provides a method for increasing expression levels of a Navα subunit selected from the group consisting of Nav1.1α, Nav1.2α, Nav1.3α, Nav1.4α, Nav1.5α, Nav1.6α, Nav1.7α, Nav1.8α, or Nav1.9α subunits in a host cell comprising co-expressing with the Navα subunit one or more sodium channel Navβ subunits selected from the group consisting Navβ1, Navβ2, or Navβ3 in a host cell, wherein the subunits are expressed in the host cell by introducing a voltage-gated sodium channel expression system disclosed herein into the host cell.

Furthermore, the present invention also provides a method for identifying an inhibitor of voltage-gated sodium channel activity (e.g., sodium flux) comprising expressing a voltage-gated sodium channel disclosed herein in a host cell as described herein, contacting the voltage-gated sodium channel with a candidate inhibitor, and determining whether the voltage-gated sodium channel exhibits lower activity in the presence of the candidate inhibitor relative to activity in the absence of the candidate inhibitor wherein the candidate inhibitor is identified as a voltage-gated sodium channel inhibitor if said lower activity is observed. In specific embodiments, the voltage-gated sodium channel activity is sodium flux. In specific embodiments, sodium flux is measured by patch-clamp assay, competition binding assay, or FLIPR® membrane potential assay.

### BRIEF DESCRIPTION OF THE DRAWINGS

FIG. 1 shows the proposed structure of human Nav1.7α (huNavα). The drawing shows a huNav1.7α model viewed from top/extracellular (top left panel) and side through cytoplasmic membrane (top right panel) wherein the extracellular space is above the sideview of the cytoplasmic membrane and the intracellular space is below the side view of the cytoplasmic membrane. Nav1.7α structural topology viewed from extracellular side (bottom panel) shown with Navβ1, Navβ2, and Navβ3 subunits.

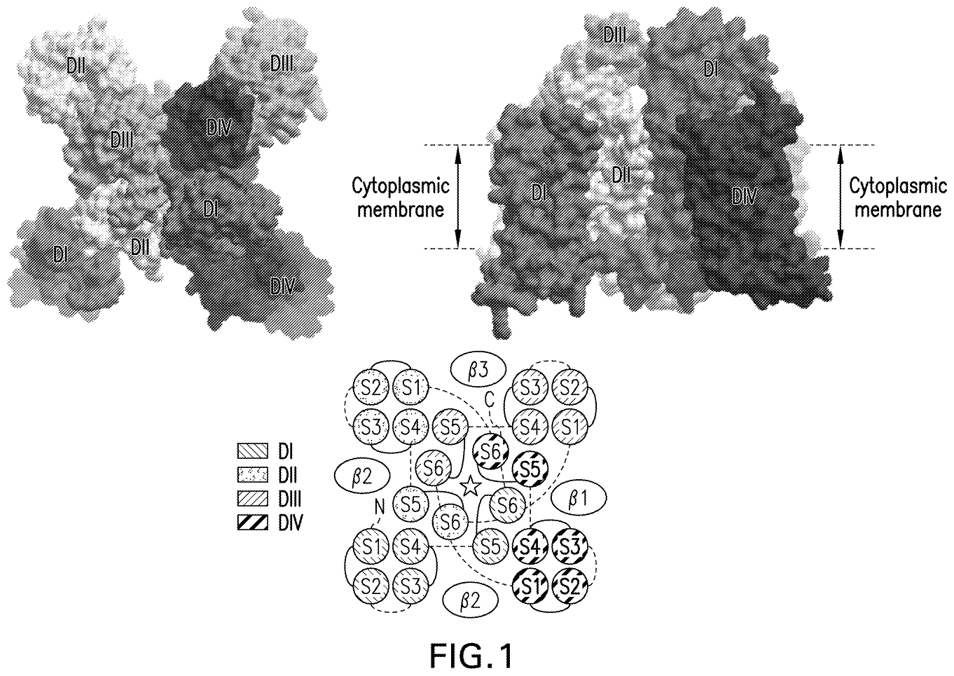

FIG. 2 shows a schematic diagram of human Nav1.7α. VSD=voltage sensing domain; PM=pore module; D=domain; S=transmembrane segment.

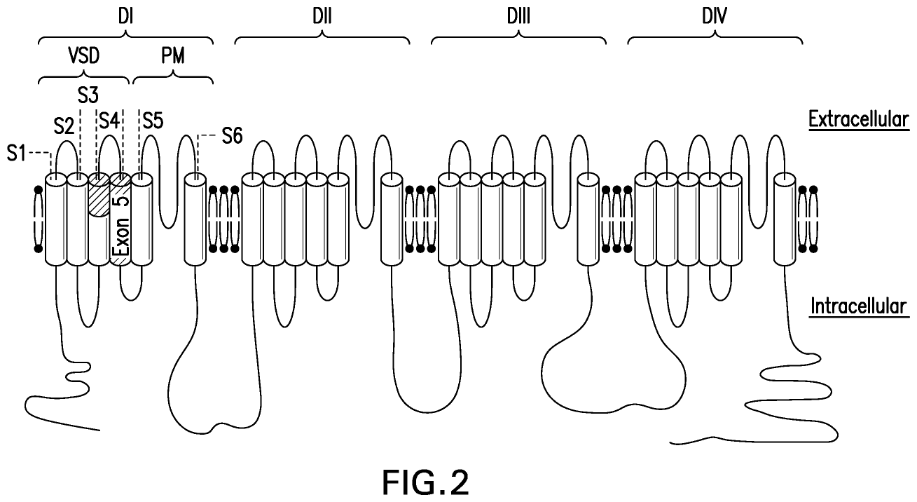

### DETAILED DESCRIPTION OF THE INVENTION

### Definitions

As used herein, the term “nucleotides” refer to organic molecules comprising a nucleoside and one to three phosphate diesters. Nucleotides serve as monomeric units of the nucleic acid polymers deoxyribonucleic acid (DNA) and ribonucleic acid (RNA). The nucleoside comprising a nucleotide is a nucleobase linked to a deoxyribose to provide a nucleotide that is deoxynucleotide for DNA or a ribose to provide a nucleotide for RNA. The four nucleobases comprising a nucleotide are guanine (G), cytosine (C), adenine (A), and thymine (T)

As used herein, a “polynucleotide” or “nucleic acid” is deoxynucleic acid (DNA) polymer comprising deoxyribonucleotides or a ribonucleic acid (RNA) polymer comprising ribonucleotides. The nucleotides comprising DNA or RNA are typically selected from guanine (G), cytosine (C), adenine (A), and thymine (T), and analogs thereof.

As used herein, a “nucleotide sequence” is a succession of nucleotides signified by a series of a set of five different letters that indicate the order of nucleotides forming alleles within a DNA (using GACT) or RNA (GACU) molecule. By convention, sequences are usually presented from the 5′ end to the 3′ end. For DNA, the sense strand is used. Because nucleic acids are normally linear (unbranched) polymers, specifying the sequence is equivalent to defining the covalent structure of the entire molecule. For this reason, the nucleic acid sequence is also termed the primary structure.

As used herein, an “amino acid sequence” is a series of two or more amino acids. As used herein, a “Protein”, “peptide”, “polyprotein”, or “polypeptide” is a contiguous string of two or more amino acids. Typically, a polypeptide is a sequence of amino acids that is 41 amino acids or more amino acids, a protein may comprise one or more polypeptides, and a peptide is an amino acid sequence that is 40 amino acids or less.

As used herein, the terms “isolated polynucleotide” or “isolated polypeptide” include a polynucleotide (e.g., RNA or DNA) or a polypeptide, respectively, which are partially (to any degree) or fully separated from other components that are normally found in cells or in recombinant DNA expression systems. These components include, but are not limited to, cell membranes, cell walls, ribosomes, polymerases, serum components and extraneous genomic sequences. An isolated polynucleotide or polypeptide will, in an embodiment of the invention, be an essentially homogeneous composition.

As used herein, a polynucleotide comprises a nucleotide sequence comprising an open reading frame (“ORF”) encoding one or more polypeptides, which may be “operably linked” to transcription and/or translation regulatory sequences, including promoters, internal ribosome entry sites (IRES) and other ribosome binding site sequences, enhancers, response elements, suppressors, signal sequences, transcription termination sequences, polyadenylation sequences, introns, 5′- and 3′-non-coding regions, and the like.

In general, transcription regulatory sequences include but are not limited to promoters, transcription enhancer sequences, response elements, transcription termination sequences and polyadenylation sequences.

In general, translation regulatory sequences include but are not limited to ribosome entry sites and other ribosome binding sequences, and translation termination sequences comprising one or more translation stop codons.

In general, a “promoter” or “promoter sequence” is a DNA regulatory region capable of binding an RNA polymerase in a cell (e.g., directly or through other promoter-bound proteins or substances) and initiating transcription of a coding sequence (open reading frame (“ORF”). A promoter sequence is, in general, linked at its 3′ terminus to a transcription initiation site and extends upstream in the 5′ direction to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding RNA polymerase. The promoter may be operably associated with other expression control sequences, including enhancer and repressor sequences, or with a polynucleotide of the present invention. Promoters which may be used to control gene expression include, but are not limited to, cytomegalovirus (CMV) promoter (U.S. Pat. Nos. 5,385,839 and 5,168,062), the SV40 early promoter region (Benoist et al., (1981) Nature 290:304-310), the promoter contained in the 3′ long terminal repeat of Rous sarcoma virus (Yamamoto et al., (1980) Cell 22:787-797), the herpes thymidine kinase promoter (Wagner et al., (1981) Proc. Natl. Acad. Sci. USA 78:1441-1445), the regulatory sequences of the metallothionein gene (Brinster et al., (1982) Nature 296:39-42); prokaryotic expression vectors such as the β-lactamase promoter (Villa-Komaroff et al., (1978) Proc. Natl. Acad. Sci. USA 75:3727-3731), or the tac promoter (DeBoer et al., (1983) Proc. Natl. Acad. Sci. USA 80:21-25); see also “Useful proteins from recombinant bacteria” in Scientific American (1980) 242:74-94; and promoter elements from yeast or other fungi such as the Gal 4 promoter, the ADC (alcohol dehydrogenase) promoter, PGK (phosphoglycerol kinase) promoter or the alkaline phosphatase promoter.

As used herein, the terms “express” and “expression” mean allowing or causing the information in a gene, RNA sequence, or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

As used herein, the term “vector” includes a vehicle (e.g., a plasmid or viral vector) by which a DNA or RNA polynucleotide may be introduced into a host cell, so as to transform the host cell and, optionally, promote expression and/or replication of the introduced sequence.

As used herein, the term “host cell” includes any cell of any organism that is isolated, selected, modified, transfected, transformed, grown, or used or manipulated in any way, for the production of a substance by the cell, for example the expression or replication, by the cell, of a gene, a DNA or RNA polynucleotide or a protein (e.g., sodium channel). Any cell type capable of expression of the voltage-gated sodium channel alpha subunit and beta subunit polypeptides via the expression system of the present invention can be used in the present invention as a host cell for expression of a sodium channel. Those having ordinary skill in the art can select a particular host cell line that is best suited for expressing a voltage-gated sodium channel polypeptide and selectable marker gene, e.g., via a vector. The present invention includes embodiments wherein the host cells are mammalian cells and cell lines and cell cultures derived therefrom. In particular embodiments, the cell type is a Chinese hamster ovary (CHO) cell (e.g., CHO K1, DG44 and DUXB11), Chinese hamster fibroblast (e.g., R1610), human cervical carcinoma (e.g., HELA), monkey kidney line (e.g., CVI and COS), murine fibroblast (e.g., BALBc/3T3), murine myeloma (P3X63-Ag3.653; NS0; SP2/O), hamster kidney line (e.g., HAK), murine L cell (e.g., L-929), human lymphocyte (e.g., RAJI), human kidney (e.g., 293 and 293T). Host cell lines are typically commercially available (e.g., from BD Biosciences, Lexington, Ky.; Promega, Madison, Wis.; Life Technologies, Gaithersburg, Md.) or from the American Type Culture Collection (ATCC, Manassas, Va.). Host cells also include bacterial cells (e.g., E. coli), insect cells such as Spodoptera frugiperda cells, SF-900, SF9, SF21 or Trichoplusia ni cells and mammalian cells such as, HEK293 cells, human amniocyte cells, murine macrophage J774 cells or any other macrophage cell line and human intestinal epithelial Caco2 cells. In an embodiment of the invention, a host cell is a lower eukaryotic or fungal cell, e.g., a yeast cell such as a glycoengineered yeast cell that produces human-like glycosylation on expressed proteins, e.g., Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pfjperi, Pichia stiptis, Pichia methanolica, Pichia sp., Saccharomyces cerevisiae, Saccharomyces sp., Hansenula polymorpha, Kluyveromyces sp., Kluyveromyces lactis or Candida albicans.

As used herein, the term “percent identity” with respect to nucleotide and amino acid sequences refers to the number of exact nucleotide or amino acids matches between nucleotide or amino acid sequences being compared. Sequence homology, not to be confused with sequence identity, refers to the biological homology between DNA, RNA, or protein sequences, defined in terms of shared ancestry in the evolutionary history of life. The following references regarding the BLAST algorithm are herein incorporated by reference: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M., et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

As used herein, “sodium channel” refers to a complex having a Navα subunit and one or more Navβ subunits from any organism, e.g., human, mouse, monkey. Sodium channels include the Nav1.1, Nav1.2, Nav1.3, Nav1.4, Nav1.5, Nav1.6, Nav1.7, Nav1.8, or Nav1.9 sodium channels. The sodium channels include a Navα subunit and one or more Navβ subunits. Sodium channel Navα subunits include, for example, Nav1.1α, Nav1.2α, Nav1.3α, Nav1.4α, Nav1.5α, Nav1.6α, Nav1.7α, Nav1.8α, or Nav1.9α subunits. The Navβ subunits include for example, Navβ1, Navβ2, or Navβ3 subunits. In an embodiment of the invention, the Nava subunit is human NaV1.7α subunit, e.g., 5N11S splice variant, or mouse Nav1.7α subunit, e.g., splice variant. In an embodiment of the invention, the Navα subunit is a Nav1.7α subunit. In an embodiment of the invention, the Navα subunit is from a tetrodotoxin-sensitive sodium channel (e.g., a Nav1.1, Nav1.6, or Nav1.7 subunit). In an embodiment of the invention, the sodium channel comprises Navα and Navβ subunits, which are each separate polypeptides and not fusion proteins comprising two or more Navα and Navβ subunits. Sodium channels may include all subunits from the same organism or different organisms, e.g., human Navα protein subunit and one or more non-human Navβ protein subunits.

As used herein, a “lipoparticle” means a small particle of about ten nanometers to about one micrometer, comprising an external lipid bilayer, which comprises one or more viral structural proteins and one or more cellular proteins. The lipoparticle is based on retrovirus structures and enables structurally intact cellular proteins to be purified away from the cell. Briefly, when a retrovirus is produced from a cell, the protein core of the virus buds through the membrane of the cell. As a consequence, the virus becomes enwrapped by the cellular membrane. Once the membrane ‘pinches’ off, the virus particle is free to diffuse. Normally, the virus also produces its own membrane protein (Envelope) that is expressed on the cell surface and that becomes incorporated into the virus. However, if the gene for the viral membrane protein is deleted, virus assembly and budding can still occur. Under these conditions, the membrane enwrapping the virus contains on or more cellular proteins, which in the context of the present invention, a sodium channel comprising a Navα protein subunit and one or more Navβ protein subunits.

As used herein, a “2A self-cleaving peptide” or “2A cleavage peptide” is a peptide from a class of 18-22 amino acid peptides, which can induce the cleaving of a polyprotein in a cell during translation. These peptides share a core sequence motif of DXEXNPGP (SEQ ID NO: 84), and are found in a wide range of viral families. They help break apart polyproteins by causing the ribosome to fail at making a peptide bond. The cleavage is triggered by breaking the peptide bond between the P and G at the C-terminus of the viral 2A peptide, resulting in the polypeptide located upstream of the 2A peptide cleavage peptide to be attached at its C-terminal end to the G of the 2A peptide cleavage peptide while the polypeptide located downstream of the 2A peptide cleavage peptide will have an extra Proline on its N-terminal end. The exact molecular mechanism of 2A-peptide-mediated cleavage is unknown. However, it is believed the “cleavage” may involve ribosomal “skipping” of glycyl-prolyl peptide bond formation rather than true proteolytic cleavage.

### Molecular Biology

In accordance with the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

### Introduction

To increase voltage-gated sodium channel functional expression in in vitro assays, polynucleotides are provided that encode a single polyprotein comprising a Navα subunit in tandem with one or more Navβ subunits, each subunit separated from the other subunits by a cleavage peptide, for example, a virally derived self-cleaving 2A peptide sequence. The single polyprotein is encoded by a polynucleotide in which the ORF encoding the polyprotein is operably linked to transcription regulatory elements. The polypeptide, which may be a deoxyribonucleic acid (DNA) molecule, can be transcribed into a polycistronic ribonucleic acid (RNA) that can be translated into the polyprotein that is cleaved at a site within the cleavage peptide either co-translationally or post-translationally into the Navα subunit and the one or more Navβ subunits. In embodiments in which the cleavage peptide is a 2A self-cleaving peptide, the 2A peptide results in cleavage or ribosome skipping at a Gly-Pro (GP) site within the self-cleaving peptide, thus resulting in liberated Navα and Navβ subunits. Thus, the voltage-gated sodium channel expression system may increase the total expression of Navα and Navβ subunits and/or an increase functional expression of Navα and Navβ subunits; along with methods of use thereof.

### Voltage-Gated Sodium Channel Expression System

The present invention provides a voltage-gated sodium channel expression system that exhibits enhanced or higher expression levels when expressed in a host cell. The voltage-gated sodium channel expression system provides a polynucleotide comprising an ORF that is capable of being transcribed to produce a polycistronic RNA message that can then be translated to produce a polyprotein that comprises both a Navα subunit and one or more Navβ subunits, each subunit separated from the other subunits by a cleavage peptide, and wherein the polynucleotide is operably linked to expression control elements at the 5′ and 3′ ends to provide a transcription unit. The Navα and Navβ subunits encoded by the polycistronic RNA message are not directly fused to each other. In particular embodiments, the polyprotein encoded by the polycistronic RNA message has the general structure from the N-terminus

(Navα subunit)—(cleavage peptide—Navβ subunit)ₙ,

wherein Navα protein subunit is selected from the group consisting of Navlia, Nav1.2α, Nav1.3α, Nav1.4α, Nav1.5α, Nav1.6α, Nav1.7α, Nav1.8α, or Nav1.9α subunits; each Navβ subunit is independently selected from the group consisting of Navβ1, Navβ2, Navβ3, and Nav β4 subunits with the proviso that the polyprotein comprising more than one Navβ subunit comprises no more than one copy of any one of Navβ1, Navβ2, Navβ3, or Navβ4 subunit; and n is 1, 2, 3, or 4; or

(Navβ-cleavage peptide)ₙ—(Navα subunit),

wherein Navα subunit is selected from the group consisting of Nav1.1α, Nav1.2α, Nav1.3α, Nav1.4α, Nav1.5α, Nav1.6α, Nav1.7α, Nav1.8α, or Nav1.9α subunits; each Navβ subunit is independently selected from the group consisting of Navβ1, Navβ2, Navβ3, and Navβ4 subunits with the proviso that the polyprotein comprising more than one Navβ comprises no more than one copy of any one of Navβ1, Navβ2, Navβ3, or Navβ4 subunit; and n is 1, 2, 3, or 4.

In an embodiment of the invention, the cleavage peptides that separate the subunits are viral 2A peptides that comprise a core motif comprising the amino acid sequence DXEXNPGP (SEQ ID NO: 84). In particular embodiments, the P is at the C-terminus of the viral 2A peptide. In other embodiments, the P is followed by a peptide sequence of two to 40 amino acids. For example, the P may be followed by a Histidine tag of about six to 10 H residues or a 1×, 2×, or 3× FLAG or MYC peptide.

In a particular embodiment of the invention, the cleavage peptides that separate the subunits are viral P2A peptides that comprise the amino acid sequence GSGATNFSLLKQAGDVEENPGP (SEQ ID NO: 1), and which may be encoded by a polynucleotide comprising the nucleotide sequence set forth in SEQ ID NO: 2. In particular embodiments, In other embodiments, the P is followed by a peptide sequence of two to 40 amino acids. For example, the P may be followed by a Histidine tag of about six to 10 H residues or a 1×, 2×, or 3× FLAG or MYC peptide.

The present invention includes embodiments in which the polyprotein is expressed and processed co-translationally to provide the individual Navα and Navβ subunits. The present invention further includes an embodiment wherein the polycistronic mRNA message is processed post-transcriptionally to generate individual RNA messages, each encoding a single Navα or Navβ subunit, which may then be translated from the individual RNA messages. In this embodiment, each Navα and Navβ subunit ORF is preceded at the 5′ and 3′ ends with the appropriate nucleotide sequences necessary for translation of the individual RNA messages to provide a translation unit that is separated by any other translation unit by a cleavage peptide.

In an embodiment of the invention, the polynucleotide comprising the transcription unit is within a vector, such as a plasmid or viral vector. In particular embodiments, the viral vector is derived from HIV-1, lentivirus, or Maloney Murine Leukemia (MLV) virus).

Thus, a voltage-gated sodium channel expression system of the present invention comprises a polynucleotide comprising an ORF encoding a Navα subunit and one or more Navβ subunits, each separated by a cleavage peptide. The ORF is flanked by transcription regulatory elements. Transcription of the ORF produces a polycistronic RNA message that may be translated into a single polyprotein comprising a Navα subunit and one or more Navβ subunits, each separated by a peptide comprising a cleavage peptide. During translation of the polycistronic RNA message, the cleavage peptide is cleaved at a specific site to generate individual Navα and Navβ subunits.

The voltage-gated sodium channel expression system may be transiently transfected into a host cell, or stably transfected into a host cell provided the voltage-gated sodium channel expression system further includes one or more nucleotide sequences that enable the voltage-gated sodium channel expression system to be integrated into the genome of the host cell. Methods for transfecting host cells and for integrating polynucleotides into a host cell are known in the art. The present invention thus includes a method for making a recombinant host cell comprising the voltage-gated sodium channel expression system as disclosed herein integrated into the host cell genome comprising the steps of introducing a polynucleotide comprising the voltage-gated sodium channel expression system and nucleotides that enable integration of the polynucleotide into the host cell genome under conditions that permit integration of heterologous polynucleotides into a host cell genome by homologous recombination or site-specific recombination.

In an embodiment of the invention, the method comprises introducing a circular plasmid vector comprising the voltage-gated sodium channel expression system as disclosed herein and a polynucleotide comprising a recombinase recognition site into a host cell comprising a recombinase recognition site integrated into the chromosome of the host cell and a gene encoding a recombinase that recognizes the recombination recognition sites, wherein under appropriate conditions the recombinase facilitates integration of the voltage-gated sodium channel expression system into the chromosomal genome via the recombination recognition sites. Thus, in an embodiment of the invention, the method includes the step of introducing a polynucleotide encoding the recombinase operably linked to transcription control elements into a host cell to provide a recombinant host cell capable of expressing the recombinase. In an embodiment of the invention, the recombinase is Cre and the site is LoxP comprising the nucleotide sequence set forth in SEQ ID NO: 3; or the recombinase is Flp recombinase and the site is an FRT site comprising the nucleotide sequence set forth in SEQ ID NO: 4 or SEQ ID NO: 5 (Craig. Ann. Rev. Genet. 22: 77-105 (1988); Sauer. Curr. Opin. Biotechnol. 5: 521-527 (1994)).

In an embodiment of the invention, the voltage-gated sodium channel expression system comprises a human, mouse, or rhesus monkey Navα subunit and/or one or more of human, mouse, or rhesus monkey Navβ subunits. The voltage-gated sodium channel expression system may comprise any one of the following exemplary Navα subunits encoded within the polycistronic RNA message or expressed therefrom:
(i) a human Nav1.1α subunit comprising the amino acid sequence set forth in SEQ ID NO: 6 and is encoded by a polynucleotide, which in particular embodiments, may have the nucleotide sequence set forth in SEQ ID NO: 7;
(ii) a human Nav1.2α subunit comprising the amino acid sequence set forth in SEQ ID NO: 8 and is encoded by a polynucleotide, which in particular embodiments, may have the nucleotide sequence set forth in SEQ ID NO: 9;
(iii) a human Nav1.3α subunit comprising the amino acid sequence set forth in SEQ ID NO: 10 and is encoded by a polynucleotide, which in particular embodiments, may have the nucleotide sequence set forth in SEQ ID NO: 11;
(iv) a human Nav1.4α subunit comprising the amino acid sequence set forth in SEQ ID NO: 12 and is encoded by a polynucleotide, which in particular embodiments, may have the nucleotide sequence set forth in SEQ ID NO: 13;
(v) a human Nav1.5α subunit comprising the amino acid sequence set forth in SEQ ID NO: 14 and is encoded by a polynucleotide, which in particular embodiments, may have the nucleotide sequence set forth in SEQ ID NO: 15;
(vi) a Nav1.6α subunit comprising the amino acid sequence set forth in SEQ ID NO: 16 and is encoded by a polynucleotide, which in particular embodiments, may have the nucleotide sequence set forth in SEQ ID NO: 17;
(vii) a human Nav1.7α subunit comprising the amino acid sequence set forth in SEQ ID NO: 18 and is encoded by a polynucleotide, which in particular embodiments, may have the nucleotide sequence set forth in SEQ ID NO: 19;
(viii) a human Nav1.8α subunit comprising the amino acid sequence set forth in SEQ ID NO: 20 and is encoded by a polynucleotide, which in particular embodiments, may have the nucleotide sequence set forth in SEQ ID NO: 21; and
(ix) a human Nav1.9α subunit comprising the amino acid sequence set forth in SEQ ID NO: 86 and is encoded by a polynucleotide, which in particular embodiments, may have the nucleotide sequence set forth in SEQ ID NO: 87.

The voltage-gated sodium channel expression system may comprise any one or more of the following exemplary Navβ subunit encoded within the polycistronic RNA message or expressed therefrom:
(a) a human Navβ1 subunit comprising the amino acid sequence set forth in SEQ ID NO: 22 and is encoded by a polynucleotide, which in particular embodiments, may have the nucleotide sequence set forth in SEQ ID NO: 23;
(b) a human Navβ2 subunit comprising the amino acid sequence set forth in SEQ ID NO: 24 and is encoded by a polynucleotide, which in particular embodiments, may have the nucleotide sequence set forth in SEQ ID NO: 25;
(c) a human Navβ3 subunit comprising the amino acid sequence set forth in SEQ ID NO: 26 and is encoded by a polynucleotide, which in particular embodiments, may have the nucleotide sequence set forth in SEQ ID NO: 27; and
(d) a human Navβ4 subunit comprising the amino acid sequence set forth in SEQ ID NO: 88 and is encoded by a polynucleotide, which in particular embodiments, may have the nucleotide sequence set forth in SEQ ID NO: 89.

In particular embodiments of the exemplary Navα or Navβ subunits, the Navα or Navβ subunit comprises an amino acid sequence having at least about 80-99.9% (in specific embodiments, 80, 81, 82, 83, 84, 85, 86, 87, 88, 89, 90, 91, 92, 93, 94, 95, 96, 97, 98, 99, 99.5, or 99.9%) identity to an amino acid sequence set forth in any of the amino acid sequences disclosed herein; when the comparison is performed by a BLAST algorithm wherein the parameters of the algorithm are selected to give the largest match between the respective sequences over the entire length of the respective reference sequences provided that a voltage-gated sodium channel comprising such a Navα and β subunit(s) assemble into sodium channels that maintain the ability to conduct sodium ions through a membrane compared to that of the native or wild-type Nava and Navβ protein subunits.

In particular embodiments of the exemplary Navα or Navβ subunits, the polynucleotide that encodes a Navα or Navβ subunit has at least about 80-99.9% (in specific embodiments, 80, 81, 82, 83, 84, 85, 86, 87, 88, 89, 90, 91, 92, 93, 94, 95, 96, 97, 98, 99, 99.5, or 99.9%) identity to a polynucleotide sequence set forth in any of the nucleotide sequences disclosed herein; when the comparison is performed by a BLAST algorithm wherein the parameters of the algorithm are selected to give the largest match between the respective sequences over the entire length of the respective reference sequences provided that a sodium channel comprising such a Navα and Navβ subunit(s) assemble into sodium channels that maintain the ability to conduct sodium ions through a membrane compared to that of the native or wild-type Navα and β subunits.

The voltage-gated sodium channel expression system may comprise a polynucleotide encoding a polyprotein comprising a Navα subunit and one or more Navβ1 subunits wherein the Navα subunits and the Navβ subunits may be in any order with the proviso that each subunit protein is separated from any adjacent subunit by a cleavage peptide and the polyprotein comprising more than one Navβ subunit comprises no more than one copy of any one of Navβ1, Navβ2, Navβ3, or Navβ4 subunits. For example, the order from the N-terminus may be exemplified by any one of the following structures:
Navα-x-Navβ;
Navβ-x-Navα;
Navα-x-Navβ-x-Navβ;
Navβ-x-Navβ-x-Navα;
Navβ-x-Navα-x-Navβ;
Navα-x-Navβ-x-Navβ-x-Navβ;
Navβ-x-Navβ-x-Navβ-x-Navα;
Navβ-x-Navβ-x-Navα-x-Navβ;
Navβ-x-Navα-x-Navβ-x-Navβ;
Navα-x-Navβ-x-Navβ-x-Navβ-x-Navβ;
NavβA-x-Navβ-x-Navβ-x-Navβ-x-Navα;
Navβ-x-Navβ-x-Navβ-x-Navα-x-Navβ;
Navβ-x-Navβ-x-Navα-x-Navβ-x-Navβ; or
Navβ-x-Navα-x-Navβ-x-Navβ-x-Navβ;

wherein the Navα subunit is selected from the group consisting of Nav1.1α, Nav1.2α, Nav1.3α, Nav1.4α, Nav1.5α, Nav1.6α, Nav1.7α, Nav1.8α, or Nav1.9α subunits; each Navβ subunit is independently selected from the group consisting of Navβ1, Navβ2, Navβ3, and Nav β4 subunit with the proviso that the polyprotein comprising more than Navβ subunit comprises no more than one copy of any one of Navβ1, Navβ2, Navβ3, or Navβ4 subunits; and x is a cleavage peptide, which in particular embodiments may be a viral 2A peptide, which in a further embodiment is a viral P2A peptide.

Exemplary polyproteins include and may be selected from any one of the following polyproteins:
(1) Navα1.1-P2A-Navβ1 comprising the amino acid sequence set forth in SEQ ID NO: 34 and which may be encoded by a polynucleotide comprising the nucleotide sequence set forth in SEQ ID NO: 58;
(2) Navα1.1-P2A-Navβ1-P2A-Navβ2 comprising the amino acid sequence set forth in SEQ ID NO: 35 and which may be encoded by a polynucleotide comprising the nucleotide sequence set forth in SEQ ID NO: 59;
(3) Navα1.1-P2A-Navβ1-P2A-Navβ2-P2A-Navβ3 comprising the amino acid sequence set forth in SEQ ID NO: 36 and which may be encoded by a polynucleotide comprising the nucleotide sequence set forth in SEQ ID NO: 60;
(4) Navα1.2-P2A-Navβ1 comprising the amino acid sequence set forth in SEQ ID NO: 37 and which may be encoded by a polynucleotide comprising the nucleotide sequence set forth in SEQ ID NO: 61;
(5) Navα1.2-P2A-Navβ1-P2A-Navβ2 comprising the amino acid sequence set forth in SEQ ID NO: 38 and which may be encoded by a polynucleotide comprising the nucleotide sequence set forth in SEQ ID NO: 62;
(6) Navα1.2-P2A-Navβ1-P2A-Navβ2-P2A-Navβ3 comprising the amino acid sequence set forth in SEQ ID NO: 39 and which may be encoded by a polynucleotide comprising the nucleotide sequence set forth in SEQ ID NO: 63;
(7) Navα1.3-P2A-Navβ1 comprising the amino acid sequence set forth in SEQ ID NO: 40 and which may be encoded by a polynucleotide comprising the nucleotide sequence set forth in SEQ ID NO: 64;
(8) Navα1.3-P2A-Navβ1-P2A-Navβ2 comprising the amino acid sequence set forth in SEQ ID NO: 41 and which may be encoded by a polynucleotide comprising the nucleotide sequence set forth in SEQ ID NO: 65;
(9) Navα1.3-P2A-Navβ1-P2A-Navβ2-P2A-Navβ3 comprising the amino acid sequence set forth in SEQ ID NO: 42 and which may be encoded by a polynucleotide comprising the nucleotide sequence set forth in SEQ ID NO: 66;
(10) Navα1.4-P2A-Navβ1 comprising the amino acid sequence set forth in SEQ ID NO: 43 and which may be encoded by a polynucleotide comprising the nucleotide sequence set forth in SEQ ID NO: 67;
(11) Navα1.4-P2A-Navβ1-P2A-Navβ2 comprising the amino acid sequence set forth in SEQ ID NO: 44 and which may be encoded by a polynucleotide comprising the nucleotide sequence set forth in SEQ ID NO: 68;
(12) Navα1.4-P2A-Navβ1-P2A-Navβ2-P2A-Navβ3 comprising the amino acid sequence set forth in SEQ ID NO: 45 and which may be encoded by a polynucleotide comprising the nucleotide sequence set forth in SEQ ID NO: 69;
(13) Navα1.5-P2A-Navβ1 comprising the amino acid sequence set forth in SEQ ID NO: 46 and which may be encoded by a polynucleotide comprising the nucleotide sequence set forth in SEQ ID NO: 70;
(14) Navα1.5-P2A-Navβ1-P2A-Navβ2 comprising the amino acid sequence set forth in SEQ ID NO: 47 and which may be encoded by a polynucleotide comprising the nucleotide sequence set forth in SEQ ID NO: 71;
(15) Navα1.5-P2A-Navβ1-P2A-Navβ2-P2A-Navβ3 comprising the amino acid sequence set forth in SEQ ID NO: 48 and which may be encoded by a polynucleotide comprising the nucleotide sequence set forth in SEQ ID NO: 72;
(16) Navα1.6-P2A-Navβ1 comprising the amino acid sequence set forth in SEQ ID NO: 49 and which may be encoded by a polynucleotide comprising the nucleotide sequence set forth in SEQ ID NO: 73;
(17) Navα1.6-P2A-Navβ1-P2A-Navβ2 comprising the amino acid sequence set forth in SEQ ID NO: 50 and which may be encoded by a polynucleotide comprising the nucleotide sequence set forth in SEQ ID NO: 74;
(18) Navα1.6-P2A-Navβ1-P2A-Navβ2-P2A-Navβ3 comprising the amino acid sequence set forth in SEQ ID NO: 51 and which may be encoded by a polynucleotide comprising the nucleotide sequence set forth in SEQ ID NO: 75;
(19) Navα1.7-P2A-Navβ1 comprising the amino acid sequence set forth in SEQ ID NO: 52 and which may be encoded by a polynucleotide comprising the nucleotide sequence set forth in SEQ ID NO: 76;
(20) Navα1.7-P2A-Navβ1-P2A-Navβ2 comprising the amino acid sequence set forth in SEQ ID NO: 53 and which may be encoded by a polynucleotide comprising the nucleotide sequence set forth in SEQ ID NO: 77;
(21) Navα1.7-P2A-Navβ1-P2A-Navβ2-P2A-Navβ3 comprising the amino acid sequence set forth in SEQ ID NO: 54 and which may be encoded by a polynucleotide comprising the nucleotide sequence set forth in SEQ ID NO: 78;
(22) Navα1.8-P2A-Navβ1 comprising the amino acid sequence set forth in SEQ ID NO: 55 and which may be encoded by a polynucleotide comprising the nucleotide sequence set forth in SEQ ID NO: 79;
(23) Navα1.8-P2A-Navβ1-P2A-Navβ2 comprising the amino acid sequence set forth in SEQ ID NO: 56 and which may be encoded by a polynucleotide comprising the nucleotide sequence set forth in SEQ ID NO: 80; and
(24) Navα1.8-P2A-Navβ1-P2A-Navβ2-P2A-Navβ3 comprising the amino acid sequence set forth in SEQ ID NO: 57 and which may be encoded by a polynucleotide comprising the nucleotide sequence set forth in SEQ ID NO: 81.

In particular embodiments of the exemplary polyproteins, the polyprotein comprises an amino acid sequence has at least about 80-99.9% (in specific embodiments, 80, 81, 82, 83, 84, 85, 86, 87, 88, 89, 90, 91, 92, 93, 94, 95, 96, 97, 98, 99, 99.5, or 99.9%) identity to an amino acid sequence set forth in any of amino acid sequences disclosed herein; when the comparison is performed by a BLAST algorithm wherein the parameters of the algorithm are selected to give the largest match between the respective sequences over the entire length of the respective reference sequences provided that a voltage-gated sodium channel comprising such Navα and Navβ subunits cleaved from the polyprotein assemble into voltage-gated sodium channels that maintain the ability to conduct sodium ions through a membrane.

In particular embodiments of the exemplary polyproteins, the polynucleotide that encodes the polyprotein has at least about 80-99.9% (in specific embodiments, 80, 81, 82, 83, 84, 85, 86, 87, 88, 89, 90, 91, 92, 93, 94, 95, 96, 97, 98, 99, 99.5, or 99.9%) identity to a polynucleotide sequence set forth in any of the nucleotide sequences disclosed herein; when the comparison is performed by a BLAST algorithm wherein the parameters of the algorithm are selected to give the largest match between the respective sequences over the entire length of the respective reference sequences provided that a voltage-gated sodium channel comprising such Navα and Navβ subunits cleaved from the polyprotein encoded by the polynucleotide assemble into voltage-gated sodium channels that maintain the ability to conduct sodium ions through a membrane.

In embodiments wherein the cleavage peptide is a viral 2A peptide, the subunit preceding the GP cleavage peptide will comprise the 2A peptide amino acid sequence upstream of the cleavage site in the cleavage peptide and have the G residue at its C-terminus and the subunit downstream of the cleavage site in the cleavage peptide will have the P residue at its N-terminus. As an example, for a polycistronic RNA message encoding a Nav1.7α+P2A+Navβ1+P2A+Navβ2+P2A+Navβ3 polyprotein wherein the viral P2A peptide comprises the amino acid sequence GSGATNFSLLKQAGDVEENPGP (SEQ ID NO: 1), during translation of the polycistronic RNA message the expressed polyprotein is being cleaved between the GP residues of the P2A peptide as the polyprotein is being synthesized. The resulting Nav1.7α will comprise the amino acid sequence GSGATNFSLLKQAGDVEENPG (SEQ ID NO: 85) at its C-terminus. The resulting Navβ1 will comprise a P at its N-terminus and the amino acid sequence GSGATNFSLLKQAGDVEENPG (SEQ ID NO: 85) at its C-terminus. The resulting Navβ2 will comprise a P at its N-terminus and the amino acid sequence GSGATNFSLLKQAGDVEENPG (SEQ ID NO: 85) at its C-terminus. The resulting Navβ3 will comprise a P at its N-terminus.

Thus, the present invention provides a human Nav1.1α, Nav1.2α, Nav1.3α, Nav1.4α, Nav1.5α, Nav1.6α, Nav1.7α, Nav1.8α, or Nav1.9α subunit comprising the amino acid sequence GSGATNFSLLKQAGDVEENPG (SEQ ID NO: 85) at its C-terminus; a human Nav1.1α, Nav1.2α, Nav1.3α, Nav1.4α, Nav1.5α, Nav1.6α, Nav1.7α, Nav1.8α, or Nav1.9α subunit comprising a P at its N-terminus; or a human Nav1.1α, Nav1.2α, Nav1.3α, Nav1.4α, Nav1.5α, Nav1.6α, Nav1.7α, Nav1.8α, or Nav1.9α subunit comprising a P at its N-terminus and comprising the amino acid sequence GSGATNFSLLKQAGDVEENPG (SEQ ID NO: 85) at its C-terminus.

Thus, the present invention provides a human Navβ1 subunit comprising the amino acid sequence GSGATNFSLLKQAGDVEENPG (SEQ ID NO: 85) at its C-terminus; a human Navβ1 subunit comprising a P at its N-terminus; or a human Navβ1 subunit comprising a P at its N-terminus and comprising the amino acid sequence GSGATNFSLLKQAGDVEENPG (SEQ ID NO: 85) at its C-terminus.

Thus, the present invention provides a human Navβ2 subunit comprising the amino acid sequence GSGATNFSLLKQAGDVEENPG (SEQ ID NO: 85) at its C-terminus; a Navβ2 subunit comprising a P at its N-terminus; or a Navβ2 subunit comprising a P at its N-terminus and comprising the amino acid sequence GSGATNFSLLKQAGDVEENPG (SEQ ID NO: 85) at its C-terminus.

Thus, the present invention provides a human Navβ3 subunit comprising the amino acid sequence GSGATNFSLLKQAGDVEENPG (SEQ ID NO: 85) at its C-terminus; a Navβ3 subunit comprising a P at its N-terminus; or a Navβ3 subunit comprising a P at its N-terminus and comprising the amino acid sequence GSGATNFSLLKQAGDVEENPG (SEQ ID NO: 85) at its C-terminus.

In a further embodiment, the present invention provides a voltage-gated sodium channel comprising a human Nav1.1α, Nav1.2α, Nav1.3α, Nav1.4α, Nav1.5α, Nav1.6α, Nav1.7α, Nav1.8α, or Nav1.9α subunit comprising the amino acid sequence GSGATNFSLLKQAGDVEENPG (SEQ ID NO: 85) at the C-terminus and/or a P at the N-terminus; and, one or more of a human Navβ1 subunit comprising a P at its N-terminus and/or comprising the amino acid sequence GSGATNFSLLKQAGDVEENPG (SEQ ID NO: 85) at the C-terminus, a human Navβ2 subunit comprising a P at the N-terminus and/or comprising the amino acid sequence GSGATNFSLLKQAGDVEENPG (SEQ ID NO: 85) at the C-terminus, and a human Navβ3 subunit comprising a P at its N-terminus and/or comprising the amino acid sequence GSGATNFSLLKQAGDVEENPG (SEQ ID NO: 85) at the C-terminus, with the proviso that a first subunit comprises only a P at its N-terminus and a second subunit comprises only the amino acid sequence GSGATNFSLLKQAGDVEENPG (SEQ ID NO: 85) at the C-terminus, and third and/or fourth subunits, if present, comprise a P at the N-terminus and the amino acid sequence GSGATNFSLLKQAGDVEENPG (SEQ ID NO: 85) at the C-terminus.

In a further embodiment, the present invention provides a voltage-gated sodium channel comprising a human Nav1.1α, Nav1.2α, Nav1.3α, Nav1.4α, Nav1.5α, Nav1.6α, Nav1.7α, Nav1.8α, or Nav1.9α subunit comprising the amino acid sequence GSGATNFSLLKQAGDVEENPG (SEQ ID NO: 85) at the C-terminus and a human Navβ1 subunit comprising a P at the N-terminus.

In a further embodiment, the present invention provides a voltage-gated sodium channel comprising a human Nav1.1α, Nav1.2α, Nav1.3α, Nav1.4α, Nav1.5α, Nav1.6α, Nav1.7α, Nav1.8α, or Nav1.9α subunit comprising the amino acid sequence GSGATNFSLLKQAGDVEENPG (SEQ ID NO: 85) at the C-terminus, a human Navβ1 subunit comprising a P at the N-terminus and comprising the amino acid sequence GSGATNFSLLKQAGDVEENPG (SEQ ID NO: 85) at the C-terminus, and a human Navβ2 subunit comprising a P at the N-terminus.

In a further embodiment, the present invention provides a voltage-gated sodium channel comprising a human Nav1.1α, Nav1.2α, Nav1.3α, Nav1.4α, Nav1.5α, Nav1.6α, Nav1.7α, Nav1.8α, or Nav1.9α subunit comprising the amino acid sequence GSGATNFSLLKQAGDVEENPG (SEQ ID NO: 85) at the C-terminus, a human Navβ1 subunit comprising a P at the N-terminus and comprising the amino acid sequence GSGATNFSLLKQAGDVEENPG (SEQ ID NO: 85) at the C-terminus, a human Navβ2 subunit comprising a P at the N-terminus and comprising the amino acid sequence GSGATNFSLLKQAGDVEENPG (SEQ ID NO: 85) at the C-terminus, and a human Navβ3 subunit comprising a P at the N-terminus.

### Expression of Voltage-gated Sodium Channels

The present invention includes methods of using the voltage-gated sodium channel expression system of the present invention for expressing a sodium channel comprising a Navα subunit and one or more β subunits. In an embodiment, the method comprises (a) introducing a voltage-gated sodium channel expression system disclosed herein encoding a Nava subunit and one or more Navβ subunits into a host cell; (b) culturing the host cell in a culture medium under conditions suitable for expressing the voltage-gated sodium channel expression system to produce the Navα subunit and the one or more Navβ subunits assembled into a sodium channel in the host cell membrane; disrupting the host cell; and obtaining the host cell membrane comprising the voltage-gated sodium channel assembled therein.

The present invention also includes methods for making a lipoparticle comprising a voltage-gated sodium channel on the surface of the particle comprising (a) introducing a voltage-gated sodium channel expression system disclosed herein encoding a Navα subunit and one or more Navβ subunits into a host cell; (b) culturing the host cell in a culture medium under conditions suitable for expressing the voltage-gated sodium channel expression system to produce the Navα subunit and the one or more Navβ subunits assembled into a voltage-gated sodium channel in the host cell membrane, which form lipoparticles comprising the sodium channel; and, (c) obtaining lipoparticles comprising the voltage-gated sodium channel assembled therein.

The present invention also includes methods for making a lipid-enveloped virus-like particle comprising a voltage-gated sodium channel on the surface of the particle comprising (a) introducing a voltage-gated sodium channel expression system disclosed herein encoding a Navα subunit and one or more Navβ subunits in a virus vector into a host cell, wherein the virus vector does not support production of infectious virus; (b) culturing the host cell in a culture medium under conditions suitable for expressing the voltage-gated sodium channel expression system to produce the Navα subunit and the one or more Navβ subunits assembled into a sodium channel integrated into the host cell membrane, which form lipoparticles comprising the voltage-gated sodium channel and, (c) obtaining lipoparticles comprising the voltage-gated sodium channel assembled therein.

Methods for making lipoparticles are well known in the art and may be used to make lipoparticles comprising sodium channels according to the present invention (See e.g., Balliet & Bates. J Virol. 72:671-676 (1998); Endres et al. Science. 278:1462-1464 (1997); Hoffman et al. Proc Natl Acad Sci U S A. 97:11215-11220 (2000); and, Rucker. Methods Mol Biol. 228:317-328 (2003)). In an embodiment of the invention, the method further includes the step of purifying the lipoparticles, e.g., isolating particles from the supernatant of the host cells. In an embodiment of the invention, the lipoparticles may be purified by ultracentrifugation, CsCl gradient centrifugation, sucrose gradient purification, and/or dialysis. Lipoparticles comprising a voltage-gated sodium channel inserted, embedded, or integrated therein produced according to methods disclosed are also part of the present invention. In an embodiment of the invention, viral vectors may be for example, an HIV-1 virus derived vector or a Maloney Murine Leukemia (MLV) virus.

Lipoparticles may be purified using sucrose cushions, as described Balliet, et al. (1998), J. Virol., 72:671-676; Endres, et al. (1997), Science, 278:1462-1464; Hoffman, et al. (2000), Proc. Natl. Acad. Sci. USA, 97:11215-11220; and U.S. Pat. No. 8,574,590. Lipoparticles may also be purified using a number of methods that are often used to purify retroviruses, see for example, Arthur, et al. (1998), AIDS Res Human Retroviruses, 3:S311-9; Ausubel, et al. (2001), Current Protocols in Molecular Biology; Dettenhofer, et al. (1999), J Virol, 73:1460-7; Le Doux, et al. (2001), Hum Gene Ther, 12:1611-21; O'Neil, et al. (1993), Biotechnology (N Y), 11:173-8; Pham, et al. (2001), J Gene Med, 3:188-94; Prior, et al. (1995), BioPharm, 25-35; Prior, et al. (1996), BioPharm, 22-34; Richieri, et al. (1998), Vaccine, 16:119-129; and, Yamada, et al. (2003), Biotechniques, 34:1074-8, 1080.

In an embodiment of the invention, methods for making a voltage-gated sodium channel further comprise lysing the host cell and isolating a membrane fraction from the lysate containing the plasma membrane in which the voltage-gated sodium channel is integrated. Methods for preparing such membrane extracts are well known in the art. For example, in an embodiment of the invention, the method for expressing a voltage-gated sodium channel further comprises exposing the host cells expressing the voltage-gated sodium channel to a mild detergent such as triton X-100 (e.g., after the cells have been incubated in a hypotonic solution), disrupting the cells (e.g., by mechanical disruption such as sonication), and isolating the fraction of the lysate containing the cell membranes (e.g., by centrifugation and recovery of the supernatant of the lysate).

The voltage-gated sodium channel expression system polynucleotides of the present invention may be introduced or transformed into an appropriate host cell by various techniques well known in the art, e.g., electroporation, protoplast fusion, calcium phosphate precipitation, cell fusion with enveloped DNA, microinjection, and infection with intact virus (see, e.g., Ridgway, 1973, Vectors: Mammalian Expression Vectors, Chapter 24.2, pp. 470-472, Rodriguez and Denhardt eds., Butterworths, Boston, Mass.; Graham et al., 1973, Virology 52:456; Sambrook et al., 1989, Molecular Cloning: A Laboratory Manual, Cold Spring Harbor Laboratories, New York; Davis et al., 1986, Basic Methods in Molecular Biology, Elsevier; and Chu et al., 1981, Gene 13:197).

Cells used in the present invention may be cultured according to standard cell culture techniques, e.g., they can be fixed to a solid surface or grown in suspension in a suitable cell culture medium.

### Voltage-Gated Sodium Channel Assays

The present invention provides methods for identifying inhibitors of voltage-gated sodium channels that have been produced from a voltage-gated sodium channel expression system of the present invention, e.g., by a method of the present invention, e.g., as discussed herein. In an embodiment of the invention, a method for identifying a voltage-gated sodium channel inhibitor is provided that comprises: (a) expressing the voltage-gated sodium channel using a voltage-gated sodium channel expression system of the present invention according to an embodiment disclosed herein, (b) contacting the voltage-gated sodium channel integrated in a membrane with a candidate inhibitor under conditions supporting voltage-gated sodium channel activity; and (c) determining said activity wherein a reduction in the level of said activity relative to the level of activity in the absence of the candidate inhibitor identifies said candidate inhibitor as a voltage-gated sodium channel inhibitor.

The voltage-gated sodium channel polypeptide activity may be, in an embodiment of the invention, ion flux (e.g., Na⁺ flux) across a membrane or sodium channel Navβ subunit/Navα subunit binding. An inhibitor of a voltage-gated sodium channel may, thus, inhibit such activity at any detectable level (e.g., 1%, 5%, 10%, 25%, 50%, 75%, 90%, 95%, 99% or 100%, relative to activity in the absence of the inhibitor). An inhibitor of a voltage-gated sodium channel may also be characterized as a therapeutic agent for treating or preventing pain (e.g., neuropathic pain, chronic pain or pain from cancer) or epilepsy.

Inhibitors of voltage-gated sodium channel sodium flux may be determined, for example, by patch-clamp assay. Such assays are generally known in the art. For example, the present invention provides a patch clamp assay method comprising (i) expressing the voltage-gated sodium channel on the surface of a cell using a voltage-gated sodium channel expression system of the present invention, (ii) immobilizing the cell on the surface of a substrate such that the cell covers and seals an aperture on the substrate wherein one ionic solution contacts the cell surface on one side of the aperture and a separate ionic solution contacts the cell surface on the other side of the aperture; and (iii) determining electrical current across the cell. In this embodiment of the invention, current is determined in the presence and absence of a candidate inhibitor wherein a reduction in current in the presence of the candidate inhibitor (e.g., relative to current in the absence of the candidate inhibitor) indicates that the candidate inhibitor is a sodium channel inhibitor.

In an embodiment of the invention, the present invention comprises a method for identifying a voltage-gated sodium channel inhibitor with a competitive binding assay that comprises (i) providing a host cell comprising a voltage-gated sodium channel expression system of the present invention wherein the host cell expresses the voltage-gated sodium channel which then assembles into the plasma membrane of the host cell or into the outer surface of the membrane of a lipoparticle or in a membrane extract prepared from the host cell; (ii) contacting the voltage-gated sodium channel with a known voltage-gated sodium channel inhibitor or binder (e.g., tetrodotoxin, GpTx-1, ProTx-I or ProTx-II, lacosamide, a mu-conotoxin, an anti-sodium channel antibody, lidocaine, carbamazepine) and with a candidate inhibitor; and (iii) determining whether binding of the known voltage-gated sodium channel inhibitor is reduced in the presence of the candidate inhibitor (e.g., relative to known voltage-gated sodium channel inhibitor binding in the absence of the candidate inhibitor); wherein said reduction indicates that the candidate inhibitor is a voltage-gated sodium channel inhibitor. In an embodiment of the invention, the known voltage-gated sodium channel inhibitor is detectably labeled, for example, with a radiolabel, such as ³H, or fluorescent moiety.

In an embodiment of the invention, the present invention comprises a method for identifying a voltage-gated sodium channel inhibitor with a FLIPR® (fluorometric imaging plate reader) assay that makes use of a membrane potential indicator dye such as DiBAC4(3) (bis-(1,3-dibutylbarbituric acid)-trimethine oxonol). Distribution of the membrane potential indicator dye (e.g., DiBAC4(3)) across the cell membrane is dependent on the membrane potential. With depolarization, the membrane potential indicator dye further partitions into the cell, leading to an increase in fluorescence. Conversely hyperpolarization results in membrane potential indicator dye extrusion and thus, a decrease in fluorescence. In an embodiment of the invention, the method comprises (i) expressing the voltage-gated sodium channel on the surface of a cell, using a voltage-gated sodium channel expression system of the present invention; (ii) monitoring the fluorescence of a cell expressing the voltage-gated sodium channel on the surface of the cell in the presence of a membrane potential indicator dye (e.g., DiBAC4(3)) and in the presence of a candidate inhibitor of the sodium channel; wherein, greater fluorescence of the cell in the presence of the candidate inhibitor (e.g., relative to fluorescence in the absence of the candidate inhibitor) indicates that the candidate inhibitor is a voltage-gated sodium channel inhibitor.

A voltage-gated sodium channel inhibitor may be a small molecule or voltage-gated sodium channel binder. A voltage-gated sodium channel binder may be a human or humanized antibody, a monoclonal antibody, a labeled antibody, a bivalent antibody, a polyclonal antibody, a bispecific antibody, a chimeric antibody, a recombinant antibody, an anti-idiotypic antibody, a humanized antibody, a bispecific antibody, or a heavy chain antibody,

A voltage-gated sodium channel binder may be an antibody fragment such as a camelized single domain antibody, an immunoglobulin single variable domain (ISVD), a VHH, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody, which may be linked to an immunoglobulin constant region, e.g., a kappa or lambda light chain, gamma-1 heavy chain, gamma-2 heavy chain, gamma-3 heavy chain or gamma-4 heavy chain.

The voltage-gated sodium channel binder may bind an epitope on an extracellular portion of the voltage-gated sodium channel and comprise a continuous or discontinuous region on the Navα subunit or Navβ subunit or a discontinuous region that spans both the Navα and Navβ subunits.

### Use of the Voltage-Gated Sodium Channel Expression System to Identify an Antibody that Specifically Binds an Epitope of a Sodium Channel

Sodium channels expressed using the voltage-gated sodium channel expression system of the present invention may be used to immunize a host animal (e.g., non-human animal, rabbit, mouse, rat, dromedary, camel or llama) for the purposes of generating an antibody or antigen-binding fragment thereof that specifically binds to an epitope of the voltage-gated sodium channel. The epitope may be an extracellular portion of the voltage-gated sodium channel and comprise a continuous or discontinuous region on the Navα or Navβ subunit or a discontinuous region that spans both the Navα and Navβ subunits.

Thus, the present invention provides a method for immunizing a host animal with a voltage-gated sodium channel produced by a host cell expressing the voltage-gated sodium channel expression system of the present invention to produce an antibody or antigen-binding fragment thereof that binds specifically to an epitope of the voltage-gated sodium channel. In one embodiment, the method for producing the antibody or antigen-binding fragment thereof comprises transfecting a host cell with the voltage-gated sodium channel expression system of the present invention to provide a host cell comprising the sodium channel expression system that expresses a viral structural protein as disclosed herein; incubating the host cell in a culture medium under conditions for expressing the voltage-gated sodium channel expression system for a time sufficient for the host cell to produce the Navα subunit and one or more Navβ subunits and assemble them into voltage-gated sodium channels integrated into a membrane of the host cell; disrupting the host cells and obtaining membranes from the disrupted host cells or lipoparticles; and administering an amount of the membrane or lipoparticles to the host animal sufficient to elicit an immune response in the host animal that causes the host animal to produce antibodies or antigen binding fragments thereof against the voltage-gated sodium channel.

In another embodiment, the method for producing the antibody or antigen-binding fragment thereof comprises transfecting a host cell with the voltage-gated sodium channel expression system of the present invention contained within a viral vector that encodes a viral structural protein as disclosed herein to provide a host cell comprising the voltage-gated sodium channel expression system; incubating the host cell in a culture medium under conditions for expressing the voltage-gated sodium channel expression system for a time sufficient for the host cell to produce the Navα subunit and one or more Navβ subunits and assemble them into voltage-gated sodium channels integrated into the membrane of a lipoparticle; obtaining the lipoparticles from the culture medium; and administering an amount of the lipoparticles to the host animal sufficient to elicit an immune response in the host animal that causes the host animal to produce antibodies or antigen binding fragments against the voltage-gated sodium channel.

In an embodiment of the invention, a hybridoma is produced from an antibody-producing B-cell of the immunized host animal. In an embodiment of the invention, the method comprises making a voltage-gated sodium channel membrane preparation using the voltage-gated sodium channel expression system of the present invention as discussed herein, administering the voltage-gated sodium channel membrane preparation to a host animal, isolating an antibody-producing B-cell from the immunized host animal (e.g., by isolating splenocytes from the spleen of the animal) and fusing the B-cell with a myeloma cell (e.g., rat or mouse myeloma), thereby producing the hybridoma; and, optionally, isolating the antibody or antigen-binding fragment thereof from the hybridoma that binds an epitope of the voltage-gated sodium channel. In an embodiment of the invention, the hybridoma is cultured in a growth medium, such as HAT medium (i.e., medium containing hypoxanthine, aminopterin and thymidine). See e.g., Stites, et al. (eds.) Basic and Clinical Immunology (4th ed.), Lange Medical Publications, Los Altos, Calif., and references cited therein; Harlow and Lane (1988) Antibodies: A Laboratory Manual, CSH Press; Goding (1986) Monoclonal Antibodies: Principles and Practice (2d ed.), Academic Press, New York; and Kohler and Milstein (1975) in Nature 256:495-497.

In an embodiment of the invention, a membrane associated voltage-gated sodium channel obtained from a host cell expressing the voltage-gated sodium channel expression system of the present invention is used with an antibody phage display library to isolate an antibody or antigen-binding fragment thereof (e.g., ScFv, Fab or nanobody) that binds specifically to an epitope of the sodium channel.

In an embodiment of the invention, the method comprises making a voltage-gated sodium channel using the sodium channel expression system of the present invention (as discussed herein), displaying a library of phage molecules (e.g., M13 or Fd) on the surfaces of host cells (e.g., bacterial cells such as E.coli), wherein each phage displays an antibody or antigen-binding fragment thereof on its surface, and selecting the host cells displaying phages having binding specificity for an epitope of the voltage-gated sodium channel; isolating the host cell and phage from the other host cells and phages and determining the sequence of the antibody or antigen-binding fragment thereof displayed on the phage surface (e.g., by isolating phage genomic DNA and determining the sequence of the portion of the phage genome encoding the antibody or antigen-binding fragment thereof), and, optionally, isolating the antibody or fragment from the phage and/or host cell. See e.g., Methods in Molecular Biology, Antibody Phage Display Methods and Protocols, Philippa M. O'Brien & Robert Aitken (eds.), Humana Press, Inc. Totowa, NJ USA, 2002.

### Specific Embodiments of the Present Invention

1. A voltage-gated sodium channel expression system comprising a polynucleotide encoding a polycistronic RNA message comprising an open reading frame (ORF) that encodes a polyprotein comprising a voltage-gated sodium channel alpha protein (Navα) subunit and one or more voltage-gated sodium channel accessory beta protein (Navβ) subunits, wherein each of the Navα and Navβ subunits are separated from an adjacent subunit by a cleavage peptide.
2. The voltage-gated sodium channel expression system embodiment 1, wherein the Nava subunit is selected from the group consisting of Nav1.1α, Nav1.2α, Nav1.3α, Nav1.4α, Nav1.5α, Nav1.6α, Nav1.7α, Nav1.8α, and Nav1.9α subunits.
3. The voltage-gated sodium channel expression system embodiment 1, wherein each Navβ subunit is selected from the group consisting of Navβ1, Navβ2, Navβ3, and Navβ4 subunits with the proviso that the polyprotein cannot comprise more than one copy of any one of Navβ1, Navβ2, Navβ3, or Navβ4 subunit.
4. The voltage-gated sodium channel expression system embodiment 2, wherein the Nav1.1α, Nav1.2α, Nav1.3α, Nav1.4α, Nav1.5α, Nav1.6α, Nav1.7α, Nav1.8α, or Nav1.9α subunit is encoded by a polynucleotide that is at least 80% identical to a nucleotide sequence set forth in SEQ ID NOs: 7, 9, 11, 13, 15, 17, 19, 21, or 87, respectively.
5. The voltage-gated sodium channel expression system embodiment 3, wherein the Navβ1, Navβ2, Navβ3, or Navβ4 subunit is encoded by a polynucleotide that is at least 80% identical to a nucleotide sequence set forth in SEQ ID NOs: 23, 25, 27, or 89, respectively.
6. The voltage-gated sodium channel expression system embodiment 1, wherein the cleavage peptide is a viral P2A peptide.
7. The voltage-gated sodium channel expression system embodiment 1, wherein the ORF comprises a nucleotide sequence with at least 80%, or in specific embodiments 90%, identity to a nucleotide sequence selected from the group of nucleotide sequences consisting of SEQ ID NOs: 58, 59, 60, 61, 62, 63, 64, 65, 66, 67, 68, 69, 70, 71, 72, 73, 74, 75, 76, 77, 78, 79, 80, and 81.
8. The voltage-gated sodium channel expression system embodiment 1, wherein the polyprotein has 80%, or in specific embodiments 90%, identity to a polyprotein comprising an amino acid sequence selected from the group of amino acid sequences consisting of SEQ ID Nos: 34, 35, 36, 37, 38, 39, 40, 41, 42, 43, 44, 45, 46, 47, 48, 49, 50, 51, 52, 53, 54, 55, 56, and 57.
9. A plasmid or viral vector comprising a nucleotide sequence encoding the polyprotein embodiment 8.
10. A host cell comprising the plasmid or viral vector embodiment 9.
11. A host cell comprising the voltage-gated sodium channel expression system embodiment 1.
12. A method for making lipoparticles comprising a voltage-gated sodium channel integrated into the membrane of the lipoparticle, comprising:(a) introducing into an isolated host cell a viral vector comprising a polynucleotide encoding a polycistronic RNA message comprising an open reading frame (ORF) that encodes a polyprotein comprising a voltage-gated sodium channel alpha protein (Navα) subunit and one or more voltage-gated sodium channel accessory beta protein (Navβ) subunits, wherein each of the subunits is separated from adjacent subunits by a cleavage peptide; (b) culturing the host cell in a cell culture medium under conditions favorable for (i) transcription of the polycistronic RNA message from the polynucleotide and translation of the polycistronic RNA message into a polyprotein that is cleaved at the cleavage peptides to produce isolated Navα and isolated one or more Navβ subunits, and (ii) generation of lipoparticles, wherein the isolated Navα subunit and isolated one or more Navβ subunits form a voltage-gated sodium channel integrated into the membrane of the lipoparticles, and (c) isolating the lipoparticles from the host cells and/or host cell culture medium.
13. The method embodiment 12, wherein the Navα subunit is selected from the group consisting of Nav1.1α, Nav1.2α, Nav1.3α, Nav1.4α, Nav1.5α, Nav1.6α, Nav1.7α, Nav1.8α, and Nav1.9α subunits.
14. The method embodiment 12, wherein each Navβ subunit is selected from the group consisting of Navβ1, Navβ2, Navβ3, and Navβ4 subunits and with the proviso that the polyprotein cannot comprise more than one copy of any one of Navβ1, Navβ2, Navβ3, or Navβ4 subunit.
15. The method embodiment 13, wherein the Nav1.1α, Nav1.2α, Nav1.3α, Nav1.4α, Nav1.5α, Nav1.6α, Nav1.7α, Nav1.8α, or Nav1.9α subunit is encoded by a polynucleotide that is at least 80% identical to a nucleotide sequence set forth in SEQ ID NOs: 7, 9, 11, 13, 15, 17, 19, 21, or 87, respectively.
16. The method embodiment 14, wherein the Navβ1, Navβ2, Navβ3, or Navβ4 subunit is encoded by a polynucleotide that is at least 80% identical to a nucleotide sequence set forth in SEQ ID NOs: 23, 25, 27, or 89, respectively.
17. The method embodiment 12, wherein the cleavage peptide is a viral P2A peptide.
18. The method embodiment 12, wherein the ORF comprises a nucleotide sequence with at least 80%, or in specific embodiments 90%, identity to a nucleotide sequence selected from the group of nucleotide sequences consisting of SEQ ID NOs: 58, 59, 60, 61, 62, 63, 64, 65, 66, 67, 68, 69, 70, 71, 72, 73, 74, 75, 76, 77, 78, 79, 80, and 81.
19. The method embodiment 12, wherein the polyprotein has 80%, or in specific embodiments 90%, identity to a polyprotein comprising an amino acid sequence selected from the group of amino acid sequences consisting of SEQ ID Nos: 34, 35, 36, 37, 38, 39, 40, 41, 42, 43, 44, 45, 46, 47, 48, 49, 50, 51, 52, 53, 54, 55, 56, and 57.
20. A lipoparticle comprising an external lipid bilayer; an enveloped retroviral structural protein; and one or more voltage-gated sodium channels, each sodium channel comprising a voltage-gated sodium channel alpha protein (Navα) subunit and one or more voltage-gated sodium channel accessory beta protein (Navβ) subunits, wherein said enveloped retroviral structural protein is an uncleaved gag protein that does not comprise a heterologous tag that binds to the voltage-gated sodium channel, provided that the only viral proteins in the lipoparticle are structural proteins.
21. The lipoparticle embodiment 20, wherein the Navα subunit is selected from the group consisting of Nav1.1α, Nav1.2α, Nav1.3α, Nav1.4α, Nav1.5α, Nav1.6α, Nav1.7α, Nav1.8α, and Nav1.9α subunits.
22. The lipoparticle embodiment 20, wherein each Navβ subunit is selected from the group consisting of Navβ1, Navβ2, Navβ3, and Navβ4 subunits with the proviso that the polyprotein cannot comprise more than one copy of any one of Navβ1, Navβ2, Navβ3, or Navβ4 subunit.
23. A host cell comprising one or more voltage-gated sodium channels integrated into the plasma membrane of the host cell, each voltage-gated sodium channel comprising a voltage-gated sodium channel alpha protein (Navα) subunit and one or more voltage-gated sodium channel accessory beta protein (Navβ) subunits, wherein each of the subunits is separated from adjacent subunits by a cleavage peptide, wherein the host cell further comprises a polynucleotide encoding a polycistronic RNA message comprising an open reading frame (ORF) that encodes a polyprotein comprising a Navα subunit and one or more Navβ subunits, wherein each of the subunits are separated from adjacent subunits by a cleavage peptide.
24. The host cell embodiment 23, wherein the Navα subunit is selected from the group consisting of Nav1.1α, Nav1.2α, Nav1.3α, Nav1.4α, Nav1.5α, Nav1.6α, Nav1.7α, Nav1.8α, and Nav1.9α subunits.
25. The host cell embodiment 23, wherein each Navβ subunit is selected from the group consisting of Navβ1, Navβ2, Navβ3, and Navβ4 subunits with the proviso that the polyprotein cannot comprise more than one copy of any one of Navβ1, Navβ2, Navβ3, or Navβ4 subunit.
26. The host cell embodiment 24, wherein the Nav1.1α, Nav1.2α, Nav1.3α, Nav1.4α, Nav1.5α, Nav1.6α, Nav1.7α, Nav1.8α, or Nav1.9α subunit is encoded by a polynucleotide that is at least 80% identical to a nucleotide sequence set forth in SEQ ID NOs: 7, 9, 11, 13, 15, 17, 19, 21, or 87, respectively.
27. The host cell embodiment 25, wherein the Navβ1, Navβ2, Navβ3, or Navβ4 subunit is encoded by a polynucleotide that is at least 80% identical to a nucleotide sequence set forth in SEQ ID NOs: 23, 25, 27, or 89 respectively.
28. The host cell embodiment 23, wherein the cleavage peptide is a viral P2A peptide.
29. The host cell embodiment 23, wherein the ORF comprises a nucleotide sequence with at least 80% or, in specific embodiments 90%, identity to a nucleotide sequence selected from the group of nucleotide sequences consisting of SEQ ID NOs: 58, 59, 60, 61, 62, 63, 64, 65, 66, 67, 68, 69, 70, 71, 72, 73, 74, 75, 76, 77, 78, 79, 80, and 81.
30. The host cell embodiment 23, wherein the polyprotein has 80%, or in specific embodiments 90%, identity to a polyprotein comprising an amino acid sequence selected from the group of amino acid sequences consisting of SEQ ID Nos: 34, 35, 36, 37, 38, 39, 40, 41, 42, 43, 44, 45, 46, 47, 48, 49, 50, 51, 52, 53, 54, 55, 56, and 57.
31. The host cell embodiment 23, where the host cell is a mammalian cell.
32. The host cell embodiment 31, wherein the mammalian host cell comprises a HEK cells or CHO cells.
33. A method for identifying an inhibitor of a voltage-gated sodium channel activity comprising: (a) providing a host cell comprising one or more voltage-gated sodium channels integrated into the plasma membrane of the host cell, each voltage-gated sodium channel comprising a voltage-gated sodium channel alpha protein (Navα) subunit and one or more voltage-gated sodium channel accessory beta protein (Navβ) subunits, wherein the host cell further comprises a polynucleotide encoding a polycistronic RNA message comprising an open reading frame (ORF) that encodes a polyprotein comprising a Navα subunit and one or more Navβ subunits, wherein adjacent subunits are separated by a cleavage peptide; (b) contacting the host cell with a candidate inhibitor and determining whether the voltage-gated sodium channel exhibits lower activity in the presence of the candidate inhibitor relative to activity in the absence of the candidate inhibitor wherein the candidate inhibitor is identified as a voltage-gated sodium channel inhibitor if said lower activity is observed.
34. The method embodiment 33 wherein the activity is sodium flux and wherein the sodium flux is measured by patch-clamp assay or fluorometric imaging plate reader assay.
35. The method embodiment 33, wherein the inhibitor is a voltage-gated sodium channel binder.
36. The method embodiment 35, wherein the voltage-gated sodium channel binder is a human or humanized antibody, a bivalent antibody, a bispecific antibody, a chimeric antibody, or a humanized heavy chain antibody.
37. The method embodiment 35, wherein the voltage-gated sodium channel binder is an antibody fragment.
38. The method embodiment 35, wherein the antibody fragment is a camelized single domain antibody, an immunoglobulin single variable domain (ISVD), a VHH, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody, which may be linked to an immunoglobulin constant region, e.g., a kappa or lambda light chain, gamma-1 heavy chain, gamma-2 heavy chain, gamma-3 heavy chain or gamma-4 heavy chain.
39. The method embodiment 33, wherein the Navα subunit is selected from the group consisting of Nav1.1α, Nav1.2α, Nav1.3α, Nav1.4α, Nav1.5α, Nav1.6α, Nav1.7α, Nav1.8α. and Nav1.9α subunits.
40. The host cell embodiment 33, wherein each Navβ subunit is selected from the group consisting of Navβ1, Navβ2, Navβ3, and Navβ4 subunits.
41. The method embodiment 35, wherein the voltage-gated sodium channel binder binds a continuous or discontinuous epitope on a Navα subunit selected from the group consisting of Nav1.1α, Nav1.2α, Nav1.3α, Nav1.4α, Nav1.5α, Nav1.6α, Nav1.7α, Nav1.8α. and Nav1.9α subunits; a continuous or discontinuous epitope on a Navβ subunit selected from the group consisting of Navβ1, Navβ2, and Navβ3 subunits; or, a discontinuous epitope that spans a Nava subunit and a Navβ subunit.
42. A voltage-gated sodium channel comprising (a) a human Nav1.1α, Nav1.2α, Nav1.3α, Nav1.4α, Nav1.5α, Nav1.6α, Nav1.7α, Nav1.8α, or Nav1.9α subunit comprising the amino acid sequence upstream of a GP cleavage site in a 2A cleavage peptide at the C-terminus; a human Nav1.1α, Nav1.2α, Nav1.3α, Nav1.4α, Nav1.5α, Nav1.6α, Nav1.7α, Nav1.8α, or Nav1.9α subunit comprising P and amino acid sequence of zero to 40 amino acids downstream of the GP cleavage site at the N-terminus; or a human Nav1.1α, Nav1.2α, Nav1.3α, Nav1.4α, Nav1.5α, Nav1.6α, Nav1.7α, Nav1.8α, or Nav1.9α subunit comprising P and amino acid sequence of zero to 40 amino acids downstream of the GP cleavage site at the N-terminus and comprising the amino acid sequence upstream of a GP cleavage site at its C-terminus; and, (b) at least one Navβ1, Navβ2, Navβ3, or Navβ4 subunit comprising the amino acid sequence upstream of a GP cleavage site at the C-terminus; a human Navβ1, Navβ2, Navβ3, or Navβ4 subunit comprising P and amino acid sequence of zero to 40 amino acids downstream of the GP cleavage site at the N-terminus; or a human Navβ1, Navβ2, Navβ3, or Navβ4 subunit comprising P and amino acid sequence of zero to 40 amino acids downstream of the GP cleavage site at the N-terminus and comprising the amino acid sequence upstream of GP cleavage site at the C-terminus, with the proviso that only one Navα or Navβ subunit comprises solely P and amino acid sequence of zero to 40 amino acids downstream of the GP cleavage site at the N-terminus and only one Navα or Navβ subunit comprises solely the amino acid sequence upstream of GP cleavage site at the C-terminus.

### EXAMPLES

The following information is provided for more clearly describing the present invention and should not be construed to limit the present invention. Any and all of the compositions and methods described below fall within the scope of the present invention.

### Example 1

Whole Cell Filter Binding Assay Assessment of Expression of Human Nav1.7α, Naβ1, hNavβ2, hNavβ3 in a Host Cell Expressing a Polynucleotide Encoding a Polyprotein Comprising the Human Nav1.7α, Navβ1, hNavβ2, and hNavβ3 Subunits

A DNA polynucleotide encoding a polyprotein comprising a human Nav1.7α fused at the C-terminus to a C-terminal tag comprising three tandem FLAG peptides and a 10×Histine peptide (3×FLAG-HIS10; SEQ ID NO: 82) followed by in order the human Navβ1, Navβ2, and Navβ3 in which four subunits are separated from each other by P2A peptides (hNav1.7α(3×FLAG-HIS10)+P2A+hNavβ1+P2A+hNavβ2+P2A+hNavβ3) was constructed. The construct comprises the amino acid sequence set forth in SEQ ID NO: 83.

The construct was stably integrated into Hek293 cells and a clone comprising the construct was selected, which was designated as Nav1.7-ctag-b1b2b3(PV)_clone11_Hek293, wherein “ctag” refers to the 3×FLAG-HIS10 and b1b2b3(PV) refers to the co-expression of Navβ1, Navβ2 and Navβ3 along with NaV1.7α upon cleavage of the P2A peptide in the polyprotein expressed from the construct. Cells from the clone were subjected to a radioligand—displacement assay. In brief, either 1×10⁵ or 2×10⁵ cells were incubated in the presence of a nM-affinity radiolabeled compound (Nav1.7 inhibitor) +/−excess unlabeled ligand. Following a three hour incubation period, cell:radioligand complexes were purified, microscintillent added to the complexes, and radioactivity measured on a Perkin-Elmer TopCount. Total Binding (TAL) and Non-Specific Binding (NSB) were measured as the amount of total signal in the absence or presence of cold-ligand respectively. Human Nav1.7 ligand binding was calculated as Specific Binding (TAL-NSB) and Signal:Background (TAL/NSB). As compared to a Hek293 cell line expressing high levels of Nav1.7α+P2A+hNavβ1 (Nav1.7b1_Hek293), the newly generated cell line Nav1.7-ctag-b1b2b3(PV)_clone11_Hek293 had a relative increase in Nav1.7 ligand binding of about three- to four-fold.

TABLE 1
Binding of compound to HEK293 cells expressing NaV1.7 sodium channel

| | NaV1.7-ctag- | | |
|---|---|---|---|
| Sample Name | b1b2b3(PV)_clone11_Hek293 | Nav1.7b1_Hek293 | Nav1.7b1_Hek293 |
| total cells/well | 100,000 | 100,000 | 200,000 |
| TAL | 2495 | 805 | 1412 |
| NSB | 177 | 171 | 220 |
| Specific Binding (TAL − NSB) | 2318 | 634 | 1192 |
| Signal:Background (TAL/NSB) | 14.1 | 4.7 | 6.4 |

### Example 2

Membrane Protein Filter Binding Assay Assessment of Expression of Human Nav1.2α, Navβ1 and hNavβ2 in a Host Cell Expressing a Polynucleotide Encoding a Polyprotein Comprising the Human Nav1.2α, Navβ1, and hNavβ2 Subunits

DNA encoding the P2A-based construct of hNav1.2α+P2A+hNavβ1+P2A+hNavβ2 was stably integrated into Hek293 cells (designated hNaV1.2_b1b2(PV)) and subject to a radioligand—displacement assay. In brief, 10 μg of membrane protein was incubated in the presence of a nM-affinity radiolabeled compound (NaV1.7 inhibitor) +/−excess unlabeled ligand. Following a three hour incubation, protein:radioligand complexes were purified, microscintillant added, and radioactivity measured on a Perkin-Elmer TopCount. Total Binding (TAL) and Non-Specific Binding (NSB) were measured as the amount of total signal in the absence or presence of unlabeled ligand, respectively. Human Nav1.2 ligand binding was calculated as Specific Binding (TAL-NSB) and Signal:Background (TAL/NSB).

As compared to three other hNav1.2 expressing cell lines, i.e., VB_Nav1.2b1b2_Hek293 (b1b2=co-expression of Navβ1+Navβ2 through traditional means in which the subunits are individual expressed as separate proteins), Milli_Nav1.2_Hek293 (Millipore; expressing human Nav1.2α and no human Navβ protein subunits) and Dx_NaV1.2 Hek293 (DiscoveRx; expressing human Nav1.2α subunit and no human Navβ subunits), the NaV1.2b1b2 P2A-based Hek293 cell line had a relative increase in NaV1.2α ligand binding of about two-fold.

TABLE 2
Binding of compound to HEK293 cells expressing human NaV1.2 sodium channel

| Sample Name | NaV1.2b1b2(PV)_Hek293 | VB_NaV1.2b1b2_Hek293 | Milli_NaV1.2_Hek293 | Dx_NaV1.2_Hek293 |
|---|---|---|---|---|
| MembraneProtein | 10 | 10 | 10 | 10 |
| (ug/well) | | | | |
| Specific Binding | 326 | 105 | 155 | 161 |
| (TAL − NSB) | | | | |
| Signal:Background | 6 | 3 | 3 | 2.9 |
| (TAL/NSB) | | | | |

### Example 3

Electrophysiology Measure of a Nav1.7 Sodium Channel in a Host Cell Expressing a Polynucleotide Encoding a Polyprotein Comprising the Human Nav1.7α, Navβ1, hNavβ2, and hNav/β3 Subunits

DNA encoding the P2A-based construct hNav1.7α+P2A+hNavβ1+P2A+hNavβ2+P2A+hNavβ3 (hNaV1.7_b1b2b3(PV)) was stably integrated into Hek293 cells (hNav1.7α-ctag-b1b2b3(PV)_clone11_Hek293) and subject to QPatch HTX-based electrophysiological recordings of voltage-gated sodium channel activity. In brief, a 48-well tissue culture plate was used to measure sodium current from individual cells. Maximum sodium currents in the presence of 150 mM NaCl were recorded from cells that have been successfully patch-clamped. As shown in Table 3, an increase in median mean current amplitudes were observed in the Nav1.7α-c-tag-b1b2b3(PV)_clone11_Hek293 cell line as compared to the Nav1.7β1_Hek293 cell line. This was consistent with a relative increase in NaV1.7 expression in the hNav1.7α-ctag-b1b2b3(PV)_clone11_Hek293 cell line. The results show that the polyprotein is properly cleaved into Navα and Navβ subunits that can form a functional sodium channel in the host cell membrane.

TABLE 3
Sodium channel activity of Hek293 cells expressing human Nav1.7

| | Median | Mean |
|---|---|---|
| | current | current |
| Cell Line/Condition | (nA) | (nA) |
| hNav1.7β1_Hek293 | 10.4 | 11.0 +/− 6.5 |
| hNav1.7α-ctag-b1b2b3(PV)_clone11_Hek293 | 19 | 18.7 +/− 9.0 |

Lengthy table referenced here
US20240012004A1-20240111-T00001
Please refer to the end of the specification for access instructions.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

LENGTHY TABLES
The patent application contains a lengthy table section. A copy of the table is available in electronic form from the USPTO web site (https://seqdata.uspto.gov/?pageRequest=docDetail&DocID=US20240012004A1). An electronic copy of the table will also be available from the USPTO upon request and payment of the fee set forth in 37 CFR 1.19(b)(3).

1. A voltage-gated sodium channel expression system comprising a polynucleotide encoding a polycistronic RNA message comprising an open reading frame (ORF) that encodes a polyprotein comprising a voltage-gated sodium channel alpha protein (Navα) subunit and one or more voltage-gated sodium channel accessory beta protein (Navβ) subunits, wherein each of the Navα and Navβ subunits are separated from an adjacent subunit by a cleavage peptide.

2. The voltage-gated sodium channel expression system of claim 1, wherein the Navα subunit is selected from the group consisting of Nav1.1α, Nav1.2α, Nav1.3α, Nav1.4α, Nav1.5α, Nav1.6α, Nav1.7α, Nav1.8α, and Nav1.9α subunits.

3. The voltage-gated sodium channel expression system of claim 1, wherein each Navβ subunit is selected from the group consisting of Navβ1, Navβ2, Navβ3, and Navβ4 subunits with the proviso that the polyprotein cannot comprise more than one copy of any one of Navβ1, Navβ2, Navβ3, or Navβ4 subunit.

4. The voltage-gated sodium channel expression system of claim 2, wherein the Nav1.1α, Nav1.2α, Nav1.3α, Nav1.4α, Nav1.5α, Nav1.6α, Nav1.7α, Nav1.8α, or Nav1.9α subunit is encoded by a polynucleotide that is at least 80% identical to a nucleotide sequence set forth in SEQ ID NOs: 7, 9, 11, 13, 15, 17, 19, 21, or 87, respectively.

5. The voltage-gated sodium channel expression system of claim 3, wherein the Navβ1, Navβ2, Navβ3, or Navβ4 subunit is encoded by a polynucleotide that is at least 80% identical to a nucleotide sequence set forth in SEQ ID NOs: 23, 25, 27, or 89, respectively.

6. The voltage-gated sodium channel expression system of claim 1, wherein the cleavage peptide is a viral P2A peptide.

7-32. (canceled)

33. A method for identifying an inhibitor of a voltage-gated sodium channel activity comprising:
(a) providing a host cell comprising one or more voltage-gated sodium channels integrated into the plasma membrane of the host cell, each voltage-gated sodium channel comprising a voltage-gated sodium channel alpha protein (Navα) subunit and one or more voltage-gated sodium channel accessory beta protein (Navβ) subunits, wherein the host cell further comprises a polynucleotide encoding a polycistronic RNA message comprising an open reading frame (ORF) that encodes a polyprotein comprising a Nava subunit and one or more Navβ subunits, wherein adjacent subunits are separated by a cleavage peptide;
(b) contacting the host cell with a candidate inhibitor and determining whether the voltage-gated sodium channel exhibits lower activity in the presence of the candidate inhibitor relative to activity in the absence of the candidate inhibitor wherein the candidate inhibitor is identified as a voltage-gated sodium channel inhibitor if said lower activity is observed.

34. The method of claim 33 wherein the activity is sodium flux and wherein the sodium flux is measured by patch-clamp assay or fluorometric imaging plate reader assay.

35. The method of claim 33, wherein the inhibitor is a voltage-gated sodium channel binder.

36. The method of claim 35, wherein the voltage-gated sodium channel binder is a human or humanized antibody, a bivalent antibody, a bispecific antibody, a chimeric antibody, or a humanized heavy chain antibody.

37. The method of claim 35, wherein the voltage-gated sodium channel binder is an antibody fragment.

38. The method of claim 35, wherein the antibody fragment is a camelized single domain antibody, an immunoglobulin single variable domain (ISVD), a VHH, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody, which may be linked to an immunoglobulin constant region, e.g., a kappa or lambda light chain, gamma-1 heavy chain, gamma-2 heavy chain, gamma-3 heavy chain or gamma-4 heavy chain.

39. The method of claim 33, wherein the Navα subunit is selected from the group consisting of Nav1.1α, Nav1.2α, Nav1.3α, Nav1.4α, Nav1.5α, Nav1.6α, Nav1.7α, Nav1.8α. and Nav1.9α subunits.

40. The host cell of claim 33, wherein each Navβ subunit is selected from the group consisting of Navβ1, Navβ2, Navβ3, and Navβ4 subunits.

41. The method of claim 35, wherein the voltage-gated sodium channel binder binds a continuous or discontinuous epitope on a Navα subunit selected from the group consisting of Nav1.1α, Nav1.2α, Nav1.3α, Nav1.4α, Nav1.5α, Nav1.6α, Nav1.7α, Nav1.8α. and Nav1.9α subunits; a continuous or discontinuous epitope on a Navβ subunit selected from the group consisting of Navβ1, Navβ2, and Navβ3 subunits; or, a discontinuous epitope that spans a Navα subunit and a Navβ subunit.

42. A voltage-gated sodium channel comprising
(a) a human Nav1.1α, Nav1.2α, Nav1.3α, Nav1.4α, Nav1.5α, Nav1.6α, Nav1.7α, Nav1.8α, or Nav1.9α subunit comprising the amino acid sequence upstream of a GP cleavage site in a 2A cleavage peptide at the C-terminus; a human Nav1.1α, Nav1.2α, Nav1.3α, Nav1.4α, Nav1.5α, Nav1.6α, Nav1.7α, Nav1.8α, or Nav1.9α subunit comprising P and the amino acid sequence of zero to 40 amino acids downstream of the GP cleavage site at the N-terminus; or a human Nav1.1α, Nav1.2α, Nav1.3α, Nav1.4α, Nav1.5α, Nav1.6α, Nav1.7α, Nav1.8α, or Nav1.9α subunit comprising P and the amino acid sequence of zero to 40 amino acids downstream of the GP cleavage site at the N-terminus and comprising the amino acid sequence upstream of a GP cleavage site at its C-terminus; and,
(b) at least one Navβ1, Navβ2, Navβ3, or Navβ4 subunit comprising the amino acid sequence upstream of a GP cleavage site in a 2A cleavage peptide at the C-terminus; a human Navβ1, Navβ2, Navβ3, or Navβ4 subunit comprising P and amino acid sequence of zero to 40 amino acids downstream of the GP cleavage site at the N-terminus; or a human Navβ1, Navβ2, Navβ3, or Navβ4 subunit comprising P and amino acid sequence of zero to 40 amino acids downstream of the GP cleavage site at the N-terminus and comprising the amino acid sequence upstream of the GP cleavage site at the C-terminus, with the proviso that only one Navα or Navβ subunit comprises solely P and amino acid sequence of zero to 40 amino acids downstream of the GP cleavage site at the N-terminus and only one Navα or Navβ subunit comprises solely the amino acid sequence upstream of GP cleavage site at the C-terminus.
